(19) United States
(12) Patent Application Publication
(10) Pub. No.: US 2017/0226555 A1
(43) Pub. Date: Aug. 10, 2017

(54) THIOL-BASED DEEP EUTECTIC SOLVENT

(71) Applicant: Hoffmann-La Roche Inc., Little Falls, NJ (US)

(72) Inventors: Mara Boenitz-Dulat, Tutzing (DE); Martin Schatte, Karlsbad (DE)

(73) Assignee: Hoffmann-La Roche Inc., Little Falls, NJ (US)

(21) Appl. No.: 15/423,920

(22) Filed: Feb. 3, 2017

(30) Foreign Application Priority Data
Feb. 5, 2016 (EP) 16154372.3

(51) Int. Cl. C12P 21/06; C12N 9/64; G01N 33/532; C07K 1/02

(52) U.S. Cl. CPC C12P 21/06; C07K 1/02; C12N 9/6472; C12Y 304/2207; G01N 33/532

(57) ABSTRACT

A deep eutectic solvent consisting of (2-hydroxyethyl) trimethyl ammonium chloride and dithiothreitol in a molar ratio of from 1:2 to 1:3 and from 0% to 10% co-solvent, and methods of enzymatic production of polypeptides using the deep eutectic solvent.

### CROSS REFERENCE TO RELATED APPLICATIONS

This Application is entitled to the priority of 16/154,372.3 filed on Feb. 5, 2016, the disclosure of which are incorporated herein by reference.

Herein is reported a thiol-based deep eutectic solvent and uses thereof.

### BACKGROUND OF THE INVENTION

Deep eutectic solvents (DESs) are water free solutions with melting points below 100° C. DESs are based on hydrogen bonds. In DES the properties of water and organic solvents are combined. Most DESs are liquid at suitable temperatures for biocatalysis. DESs are more hydrophobic than water however in some cases they do not lead to hydrophobicity induced inactivation of the biocatalyst (Gorke, J., et al., Biotechnol. Bioprocess E 15 (2010) 40-53.). Additionally deep eutectics solvents can be produced water free and are therefore inert for hydrolysis. Ionic liquids have similar properties as DES, but are more difficult to produce and more harmful to the environment. Proteases have shown activity in DES (Zhao, H., et al., J. Mol. Catal. B-Enzym 72 (2011) 163-167).

Sortase A (SrtA) is a membrane bound enzyme which attaches proteins covalently to the bacterial cell wall. The specific recognition motif on the SrtA substrate is LPXTG, whereby the enzyme cleaves between the residues threonine and glycine. The recognition motif on the peptidoglycan is a pentaglycine motif. It has been shown that a triglycine and even a diglycine motif on the N-terminus is sufficient to support the SrtA reaction (Clancy, K. W., et al., Peptide science 94 (2010) 385-396). The reaction proceeds through a thioester acyl-enzyme intermediate, which is resolved by the attack of an amine nucleophile from the oligoglycine, covalently linking peptidoglycan to a protein substrate and regenerating SrtA. SrtA can be used to covalently conjugate chemically synthetized peptides to recombinantly expressed proteins.

Applicable Sortases for technical bioconjugation are limited. The most wildly used Staphylococcus aureus Sortase A (St.au. SrtA) shows suitable conversion kinetics for technical application but has a limited substrate spectrum, only recognizing LPXTG sortase-motives. The St.au. SrtA (Sa-SrtA), that lacks the N-terminal membrane-anchoring motif, has been used for cell-surface protein labeling, covalent protein immobilization and incorporation of novel functionality into proteins. For orthogonal/dual labeling strategies, sortases with new substrate spectra are needed. The same holds true for standard sortase mediated bioconjugation approaches where a LPXTG motive in the product has e.g. negative effects on its structure and/or function. Therefore sortases with recognition sequences different from LPXTG would be of high value. The Staphylococcus pyogenes SrtA (St.py. SrtA) recognizes a LPXTA sortase-motives, however the conversion kinetic parameter of the enzyme turn it in to a not suitable sortase on a technical scale.

Sortases that accept sortase-motives different from LPXTG are reported in literature. Thereunder are wild-types e.g. Sortase A from Streptococcus pyogenes (St.py. SrtA) and Sortase A from Clostridium difficile (Cl.di. SrtA) as well as engineered sortase. Beside the St.py. SrtA none of the reported sortase recognizes a LPXTA motif (see e.g. van Leeuwen, H. C., et al., FEBS Lett. 588 (2014) 4325-4333; Don, B. M., et al., Proc. Natl. Acad. Sci. USA 111 (2014) 13343-13348; Bentley, M. L., et al., J. Biol. Chem. 282(2007) 6571-6581; Race, P. R., et al., J. Biol. Chem. 284 (2009) 6924-33; Antos, J. M., et al., J. Am. Chem. Soc. 131 (2009) 10800-10801).

Sortase reactions are performed in aquatic solutions which has several drawbacks. One is that many compounds have a low solubility in water—this is especially true for many fluorophores. Additionally sortases have a very low Km making hydrophobic substrate not accessible for sortase reactions (Chen, I., et al., Proc. Natl. Acad. Sci. USA 108 (2011) 11399-11404). However, antibody/binding-proteins linked to a fluorophore are probably the most important application in bioconjugation (Shreve, P. and Aisen, A. M., Magnetic resonance in medicine: official journal of the Society of Magnetic Resonance in Medicine/Society of Magnetic Resonance in Medicine 3 (1986) 336-340; Drapkin, R. L., et al., Am. J. Hematol. 7 (1979) 163-172). Another issue is the enzyme intermediate which is formed during the reaction. It can be hydrolyzed which leads to product loss. This makes water free and organic solvents an interesting alternative to water. However sortases are not stable in organic solvents e.g. more than 20% Dimethyl sulfoxide (DMSO) diminishes sortase activity (Pritz, S. (2008) Enzymatische Ligation von Peptiden, Peptidnucleinsauren and Proteinen).

In WO 2010/087994 methods for ligation and uses thereof are reported. Recombinant approaches to IgG-like bispecific antibodies are reported by Marvin, J. S., et al. (Acta Pharmacol. Sinica 26 (2005) 649-658). In WO 2013/003555 the use of sortases to install click chemistry handles for protein ligation is reported.

Strijbis, K. et al (Traffic 13 (2012) 780-789) report protein ligation in living cells using sortase. It has been stated by them that the Ca²⁺-dependent S. aureus sortase A is not functional intracellularly, but that the Ca²⁺-independent S. pyogenes sortase A is functional in the cytosol and endoplasmic reticulum (ER) lumen of both Saccharomyces cerevisiae and mammalian HEK293T cells.

Levary, D. A., et al., report protein-protein fusion catalyzed by Sortase A (PLOS ONE 6 (2011)). Engineering of an anti-epidermal growth factor receptor antibody to single chain format and labeling by sortase A-mediated protein ligation is reported by Madej, M. P., et al. (Biotechnol. Bioeng. 109 (2012) 1461-1470). Ta, H. T., et al., report enzymatic single-chain antibody tagging as a universal approach to targeted molecular imaging and cell homing in cardiovascular diseases (Cir. Res. 109 (2011) 365-373). Popp, M., et al., report making and breaking peptide bonds—protein engineering using sortase (Angew. Chem. Int. Ed. Eng. 50 (2011) 5024-5032). Engineered proteins with high affinity for DOTA chelates are reported in WO 2010/099536.

Different efforts to block the revers reactions of Sortase have been reported. Yamamura, Y., et al. (Chem. Commun. 47 (2011) 4742-4744) reported enhancement of sortase A-mediated protein ligation by inducing a beta-hairpin structure around the ligation site by introducing a β-hairpin around the recognition site of the substrate.

Sorting of LPXTG peptides by archetypal sortase A, role of invariant substrate residues in modulating the enzyme dynamics and conformational signature of a productive substrate was reported by Biswas, T., et al. (Biochem. 53 (2014) 2515-2524).

Li, Y. M., et al. report irreversible site-specific hydrazinolysis of proteins by use of Sortase (Angew. Chem. Int. Ed. Engl. 53 (2014) 2198-2202). Ling and co-workers showed the introduction of a thioester via a sortase (Ling, J. J. J., et al., J. Am. Chem. Soc. 134 (2012) 10749-10752). Lindberg, D., et al. (J. Biotechnol. 147 (2010) 169-171) reported deep eutectic solvents (DESs) are viable co-solvents for enzyme-catalyzed epoxide hydrolysis. Gorke, J. T., et al. reported hydrolase-catalyzed biotransformations in deep eutectic solvents (Chem. Commun. (2008) 1235-1237).

Bellucci, J. J., et al. report the use of lysine as nucleophile (Angew. Chem. Int. Ed. Engl. 53 (2014) 1-6).

In U.S. Pat. No. 8,247,198 enzymatic processing in deep eutectic solvents is reported.

In WO 2002/26701 ionic liquids and their use as solvents are reported. It reports ionic compounds with a freezing point of up to 100° C. by the reaction of one amine salt, such as choline chloride, with an organic compound capable of forming a hydrogen bond with the ion of the amine salt, such as urea, wherein the molar ratio is from 1:1.5 to 1:2.5.

In WO 2000/56700 ionic liquids are reported. It reports ionic liquids having a melting point of no more than 60° C., formed by the reaction of a quaternary ammonium compound or a mixture of two or more thereof with a halide of zinc, tin or iron, or a mixture of two or more thereof.

EP2 597 099 reports a deep eutectic solvent and method for its preparation.

Kareem, M. A., et al. (J. Chem. Eng. Data 55 (2010) 4632-4637) reported phosphonium-based ionic liquids analogues and their physical properties.

In U.S. Pat. No. 7,763,768 a method for the preparation of reactive hydrogen peroxide in deep eutectic solvents is reported.

In WO 2014/131906 it is reported that it is possible to stabilize biomolecules such as RNA, DNA and proteins in biological samples such as cells, tissues, biopsies and blood using deep eutectic solvents (DES). DES mixtures can be used to fix and preserve cell morphology in biological samples such as tissue blocks, cancer biopsies and whole blood.

Smith, E. L.et al., described Deep Eutectic Solvents (DESs) and their applications (Chem. Rev. 114 (2014) 11060-11082).

Schmohl, L., et al., reported sortase-mediated ligations for the site-specific modification of proteins (Curr. Opin. Chem. Biol. 22 (2014) 122-128).

Ling, J. J., et al., reported protein thioester synthesis enabled by sortase (J. Am. Chem. Soc. 134 (2012) 10749-10752).

### SUMMARY OF THE INVENTION

It has been found that thiol-based deep eutectic solvents can be made. Such thiol-based deep eutectic solvents comprise (a) at least one hydrogen bond acceptor and (b) at least one hydrogen bond donor comprising at least i) one thiol group and ii) one hydroxy group or negatively charged oxygen atom or a salt thereof.

It has further been found that a transpeptidation reaction can be performed in a thiol-based deep eutectic solvent as reported herein. It has further been found that the reduction/absence of water is not detrimental to the reaction but suppresses hydrolysis side reaction.

One aspect as reported herein is a mixture/deep eutectic solvent comprising
a) at least one hydrogen bond acceptor, and
b) at least one aliphatic hydrogen bond donor comprising i) at least one thiol group and ii) at least one hydroxy group or negatively charged oxygen atom or a salt thereof,

wherein the molar ratio of the at least one hydrogen bond acceptor and the at least one aliphatic hydrogen bond donor is of from 2:1 to 1:10.

In one embodiment the molar ratio of the at least one hydrogen bond acceptor and the at least one aliphatic hydrogen bond donor is of from 1:0.5 to 1:4. In one embodiment the molar ratio of the at least one hydrogen bond acceptor and the at least one aliphatic hydrogen bond donor is of from 1:1 to 1:3.

In one embodiment the mixture comprises one hydrogen bond acceptor and one aliphatic hydrogen bond donor.

In one embodiment the aliphatic hydrogen bond donor comprises 1, 2, 3, 4, or 5 thiol groups. In one embodiment the aliphatic hydrogen bond donor comprises 1 or 2 thiol groups.

In one embodiment the aliphatic hydrogen bond donor is an alkane comprising i) at least one thiol group and ii) at least one hydroxy group or negatively charged oxygen atom or a salt thereof In one embodiment the alkane is a linear or branched alkane comprising of from 2 to 6 carbon atoms. In one preferred embodiment the alkane is a linear or branched alkane comprising 2 to 4 carbon atoms.

In one embodiment the aliphatic hydrogen bond donor comprises 1 to 4 thiol groups. In one preferred embodiment the aliphatic hydrogen bond donor comprises 1 or 2 thiol groups.

In one embodiment the aliphatic hydrogen bond donor comprises 1 to 4 hydroxy groups. In one preferred embodiment the aliphatic hydrogen bond donor comprises 1 or 2 hydroxy groups.

In one embodiment the aliphatic hydrogen bond donor comprises a sulfino or sulfo group or a salt thereof.

In one preferred embodiment the aliphatic hydrogen bond donor is a linear or branched alkane comprising 2 to 4 carbon atoms comprising i) one or two thiol group and ii) one or two hydroxy groups or a sulfo group or a salt thereof

In one embodiment the aliphatic hydrogen bond donor additionally comprises 1, 2, 3, 4, or 5 functional groups. In one embodiment the functional groups are independently from each other selected from the group consisting of carboxyl group, amino group, aldehyde group or ether group.

In one embodiment the aliphatic hydrogen bond donor is selected from hydroxy-thiol compounds, thio-sulfino compounds and thio-sulfo compounds and salts thereof In one embodiment the hydrogen bond donor is selected from the group consisting of dithiothreitol, 2-mercaptoethanol, 4-mercapto-1-butanol, 1-mercapto-2-propanol and sodium 2-mercaptoethansulfonate.

In one embodiment the at least one hydrogen bond acceptor is selected from the group consisting of (2-hydroxyethyl) trimethyl ammonium salts, methyl triphenyl phosphonium salts, ethylene amine salts and tributyl amine salts. In one embodiment the salts are chlorides, bromines and hydroxides.

In one embodiment the hydrogen bond donor comprising at least one thiol group has a freezing point of 200° C. or less. In one embodiment the hydrogen bond donor has a freezing point of 85° C. or less.

Ethylene amine salts include ethylene diamine salts, diethylene triamine salts, triethylene tetramine salts, tetraethylene pentamine salts, pentaethylene hexamine salts, 2,2′, 2′-triamino triethyl amine, piperazine, aminoethyl piperazine,

In one preferred embodiment the molar ratio of the at least one hydrogen bond acceptor and dithiothreitol is about 1:2.5 to 1:3. In one embodiment the at least one hydrogen bond acceptor is choline chloride.

In one preferred embodiment the molar ratio of the at least one hydrogen bond acceptor and 2-mercaptoethanol is about 1:2. In one embodiment the at least one hydrogen bond acceptor is choline chloride.

In one preferred embodiment the molar ratio of the at least one hydrogen bond acceptor and 4-mercapto-158-butanol is about 1:2. In one embodiment the at least one hydrogen bond acceptor is choline chloride.

In one preferred embodiment the molar ratio of the at least one hydrogen bond acceptor and 1-mercapto-2-propanol is about 1:2. In one embodiment the at least one hydrogen bond acceptor is choline chloride.

In one preferred embodiment the molar ratio of the at least one hydrogen bond acceptor and sodium 2-mercaptoethansulfonate is about 1:1. In one embodiment the at least one hydrogen bond acceptor is choline chloride.

One aspect as reported herein is a method for preparing a deep eutectic solvent comprising the steps of i) combining at least one hydrogen bond acceptor and at least one hydrogen bond donor comprising at least one thiol group, and ii) heating the combination to a temperature of 60° C. or more and cooling the heated combination.

In one embodiment the heating is to a temperature of 60° C. or more and less than 200 ° C. In one embodiment the heating is to a temperature of from 100° C. to 150° C.

One aspect as reported herein is a method for the enzymatic production of a polypeptide comprising the following step

incubating
i) a first polypeptide comprising (optionally within the 100 C-terminal amino acid residues) the amino acid sequence LPXTG (SEQ ID NO: 01, wherein X can be any amino acid residue) or LPXTA (SEQ ID NO: 101, wherein X can be any amino acid residue),
ii) a second polypeptide comprising i) a glycinyl, an alaninyl, or a cysteinyl compound at its N-terminus, or ii) an oligoglycine, or oligoalanine, or a cysteine amino acid residue followed by one to three glycine or alanine amino acid residues at its N-terminus, or iii) a lysine amino acid residue within its 5 N-terminal amino acid residues, and
iii) a third polypeptide with sortase A activity,

in a deep eutectic solvent as reported herein

and thereby producing a polypeptide.

In one embodiment the third polypeptide with sortase A activity is derived from Staphylococcus aureus sortase A, or from Streptococcus pyogenes Sortase A, or from Listeria monocytogenes Sortase A.

In one embodiment the third polypeptide is derived from Staphylococcus aureus sortase A, or from Streptococcus pyogenes Sortase A, or from Listeria monocytogenes Sortase A.

In one embodiment the third polypeptide comprises the amino acid sequence of SEQ ID NO: 05, SEQ ID NO: 06, SEQ ID NO: 27, SEQ ID NO: 156, SEQ ID NO: 159, SEQ ID NO: 160 or SEQ ID NO: 161. In one preferred embodiment the third polypeptide comprises the amino acid sequence of SEQ ID NO: 05 or 06.

In one embodiment the third polypeptide comprises additionally a tag at its N- or C-terminus either conjugated directly or via an intervening linker. In one embodiment the third polypeptide is consisting of the amino acid sequence of SEQ ID NO: 05 and the C-terminal tag of SEQ ID NO: 32. In one embodiment the third polypeptide is consisting of the amino acid sequence of SEQ ID NO: 05.

In one embodiment the method is for the enzymatic conjugation of two polypeptides. In one embodiment the method is for the enzymatic transpeptidation of two polypeptides.

In one embodiment the incubating is at a temperature of from 30° C. to 40° C. In one embodiment the incubating is at a temperature of about 37 ° C.

In one embodiment the second polypeptide has at its N-terminus the amino acid sequence GGG (SEQ ID NO: 29), AAA (SEQ ID NO: 162), CGG (SEQ ID NO: 163), CAA (SEQ ID NO: 164), KGG (SEQ ID NO: 165) or KAA (SEQ ID NO: 166).

In one embodiment the first polypeptide comprises at its C-terminus the amino acid sequence LPXTG (SEQ ID NO: 01, wherein X can be any amino acid residue) or LPXTA (SEQ ID NO: 101, wherein X can be any amino acid residue). In one embodiment the first polypeptide comprises at its C-terminus the amino acid sequence LPETG (SEQ ID NO: 04) or LPETA (SEQ ID NO: 108).

In one embodiment the first polypeptide and the second polypeptide are independently of each other selected from an antibody variable domain, an antibody heavy chain Fab-fragment, an antibody Fc-region, a tag, an antibody heavy chain, an antibody light chain, and a peptide, a linker and a non-sortase motif moiety each comprising the amino acid sequence LPXTG (SEQ ID NO: 01, wherein X can be any amino acid residue) or LPXTA (SEQ ID NO: 101, wherein X can be any amino acid residue).

One aspect as reported herein is the use of a deep eutectic solvent as reported herein as solvent in an enzymatic transamidation reaction catalyzed by Sortase A.

In one embodiment of all aspects as reported herein the deep eutectic solvent comprises more than 0% and up to 10% (v/v) aqueous co-solvent. In one embodiment of all aspects as reported herein the deep eutectic solvent comprises of from 1% to 10% (v/v) aqueous co-solvent. In one embodiment the aqueous co-solvent is an aqueous buffer. In one embodiment the aqueous co-solvent is water.

### DETAILED DESCRIPTION OF THE INVENTION

The invention is at least in part based on the finding that deep eutectic solvents can be used to conduct transamidation reactions, especially sortase catalyzed reactions.

The invention is at least in part based on the finding that hardly water soluble substrates can be conjugated by sortase in deep eutectic solvents.

### I. Definitions

The term “derived from” denotes that the respective amino acid sequence comprises the same amino acid sequence, or contains amino acid sequence changes, or is a shortened variant or a fused variant of a parent amino acid sequence.

The term “comprising” when used herein expressly includes the term “consisting of”.

The term “a glycinyl, an alaninyl, or a cysteinyl compound” denotes a compound that comprises a glycine or an alanine or a cysteine amino acid residue with free alpha amino group, e.g. as NH2 or NH3+, and a carboxy group at position 1 that is in a peptide bond with another moiety, whereby the moiety can be any amino group containing moiety, such as an isolated amino acid residue, a peptide, a polypeptide, a protein, a small molecule, a dye, etc.

In the present specification and claims the numbering of the residues in an immunoglobulin heavy chain Fc-region is that of the EU index of Kabat (Kabat, E. A., et al., Sequences of Proteins of Immunological Interest, 5th ed., Public Health Service, National Institutes of Health, Bethesda, MD (1991), NIH Publication 91-3242, expressly incorporated herein by reference).

The term “aliphatic”, as used herein, denotes a non-substituted or substituted linear or branched hydrocarbon. This hydrocarbon can be completely saturated, i.e. it contains only carbon-carbon single bonds (also denoted as alkane), or it may contain one or more carbon-carbon double bonds (also denoted as alkene) or triple bonds (also denoted as alkyne) but nevertheless it is in no case comprising an aromatic moiety. For example, an aliphatic compound includes non-substituted and substituted linear or branched alkanes, alkenes and alkynes as well as hybrids thereof In certain embodiments, the straight or branched carbon atom chain has about 6 or fewer carbon atoms in its backbone. Exemplary aliphatic compounds groups include ethane n-propane, iso-propane, n-butane, sec-butane, iso-butane, and tert-butane. The term “aliphatic hydrogen bond donor comprising i) at least one thiol group and ii) at least one hydroxy group or negatively charged oxygen atom or a salt thereof” denotes an aliphatic compound that is substituted with at least one thiol group (HS—) and at least one hydroxyl group (HO—). The term “alteration” denotes the mutation, addition, or deletion of one or more amino acid residues in a parent amino acid sequence, e.g. of an antibody or fusion polypeptide comprising at least an FcRn binding portion of an Fc-region, to obtain a variant antibody or fusion polypeptide.

The term “amino acid mutation” denotes a modification in the amino acid sequence of a parent amino acid sequence. Exemplary modifications include amino acid substitutions, insertions, and/or deletions. In one embodiment the amino acid mutation is a substitution. The term “amino acid mutations at the position” denotes the substitution or deletion of the specified residue, or the insertion of at least one amino acid residue adjacent the specified residue. The term “insertion adjacent to a specified residue” denotes the insertion within one to two residues thereof. The insertion may be N-terminal or C-terminal to the specified residue.

The term “amino acid substitution” denotes the replacement of at least one amino acid residue in a predetermined parent amino acid sequence with a different “replacement” amino acid residue. The replacement residue or residues may be a “naturally occurring amino acid residue” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). In one embodiment the replacement residue is not cysteine. Substitution with one or more non-naturally occurring amino acid residues is also encompassed by the definition of an amino acid substitution herein. A “non-naturally occurring amino acid residue” denotes a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine, alpha-amino isobutyric acid (aib) and other amino acid residue analogues such as those described in Ellman, et al., Meth. Enzym. 202 (1991) 301-336. To generate such non-naturally occurring amino acid residues, the procedures of Noren, et al. (Science 244 (1989) 182) and/or Ellman, et al. (supra) can be used. Briefly, these procedures involve chemically activating a suppressor tRNA with a non-naturally occurring amino acid residue followed by in vitro transcription and translation of the RNA. Non-naturally occurring amino acids can also be incorporated into peptides via chemical peptide synthesis and subsequent fusion of these peptides with recombinantly produced polypeptides, such as antibodies or antibody fragments.

The term “amino acid insertion” denotes the incorporation of at least one additional amino acid residue into a predetermined parent amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as defined above.

The term “amino acid deletion” denotes the removal of at least one amino acid residue at a predetermined position in an amino acid sequence.

Within this application whenever an amino acid alteration is mentioned it is a deliberated amino acid alteration and not a random amino acid modification.

The term “tag” denotes a sequence of amino acid residues connected to each other via peptide bonds that has specific binding properties. In one embodiment the tag is an affinity or purification tag. In one embodiment the tag is selected from Arg-tag, His-tag, Flag-tag, 3xFlag-tag, Strep-tag, HA-tag, Nano-tag, SBP-tag, c-myc-tag, S-tag, SNUT-Tag, NusA, T7, thioredoxin, calmodulin-binding-peptide, cellulose-binding-domain, chitin-binding-domain, GST-tag, or MBP-tag (see, e.g., Amau, J., et al., Prot. Expr. Purif. 48 (2006) 1-13).

In one embodiment the tag is selected from SEQ ID NO: 07 (RRRRR), or SEQ ID NO: 08 (RRRRRR), or SEQ ID NO: 09 (HHHHHH), or SEQ ID NO: 10 (KDHLIHNVHKEFHAHAHNK), or SEQ ID NO: 11 (DYKDDDDK), or SEQ ID NO: 12 (DYKDHDGDYKDHDIDYKDDDDK), or SEQ ID NO: 13 (AWRHPQFGG), or SEQ ID NO: 14 (WSHPQFEK), or SEQ ID NO: 15 (MDVEAWLGAR), or SEQ ID NO: 16 (MDVEAWLGARVPLVET), or SEQ ID NO: 17 (MDEKTTGWRGGHVVEGLAGELEQLRARLEHHPQGQREP), or SEQ ID NO: 18 (EQKLISEEDL), or SEQ ID NO: 19 (KETAAAKFERQHMDS), or SEQ ID NO: 20 (KRRWKKNFIAVSAANRFKKISSSGAL), or SEQ ID NO: 21 (cellulose binding domain), or SEQ ID NO: 22 (cellulose binding domain), or SEQ ID NO: 23 (TNPGVSAWQVNTAYTAGQLVTYNGKTYKCLQPHTSLAGWEP SNVPALWQLQ), or SEQ ID NO: 24 (GST-tag), or SEQ ID NO: 25 (MBP-tag), or SEQ ID NO: 32 (MRGSHHHHHHGS).

An “effective amount” of an agent, e.g., a pharmaceutical formulation, refers to an amount effective, at dosages and for periods of time necessary, to achieve the desired therapeutic or prophylactic result.

The term “individual” or “subject” denotes a mammal. Mammals include, but are not limited to, domesticated animals (e.g. cows, sheep, cats, dogs, and horses), primates (e.g., humans and non-human primates such as monkeys), rabbits, and rodents (e.g., mice, rats, and hamsters). In certain embodiments, the individual or subject is a human.

The term “pharmaceutical formulation” refers to a preparation which is in such a form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “position” denotes the location of an amino acid residue in the amino acid sequence of a polypeptide. Positions may be numbered sequentially, or according to an established format, for example the EU index of Kabat for antibody numbering.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

### II. Deep Eutectic Solvents

The term “deep eutectic solvent (DES)” as used herein denotes an ionic solvent which is formed by a mixture of compounds that form an eutectic system with a melting point significant lower than that of each of its individual components. A DES is generally a mixture comprising a nitrogen or metal salt as hydrogen bond acceptor (first component) and a hydrogen bond donor (second component) forming a eutectic point at the specific applied ratio.

Deep eutectic solvents are mixtures comprising polar components (liquids or solids) that when combined have decreased melting temperatures. DESs can be produced by mixing choline chloride (ChCl) with an organic hydrogen donor, e.g. an amine, amide, alcohol, or carboxylic acid (see e.g. Abbott, A. P., et al., J. Am. Chem. Soc. 126 (2004) 9142-9147; ibid. Chem. Commun. (2003) 70-71). The DES comprises anions, cations and neutral components so that a DES has both, ionic and organic solvent properties (Zhang et al., Angew. Chem. Int. Ed. 48 (2009) 3486-3490).

Proteases have shown activity in DES (Zhao, H., et al., J. Mol. Catal. B-Enzym 72 (2011) 163-167). Likewise lipases have shown activity in DES at 50° C. with 1% (v/v) water (Zhao, H., et al., J. Mol. Catal. B-Enzymatic 85-86 (2013) 243-247) or in a solution comprising 0.135 mL 2-propanol, 1.215 mL Tris-HCl buffer (50 mmol L-1, pH 8.0) and 0-1.2mo1 L-1 of DES at 37° C. or combining 0.4-0.5 ml oil, 2.1 mL DES, 0.9 mL methanol, and 300 mg enzyme powder without addition of extra water at 50° C. (Huang, Z.-L., et al., J. Chem. Technol. Biotechnol. 89 (2014) 1975-1981) or at 40° C. to 60° C. (U.S. Pat. No. 8,247,198).

Generally DES can be made by simply warming and stirring the components for a certain time, optionally with a successive drying step. For example, DES can be prepared by a thermal treatment of the previously mixed components (see Abbott (2003) supra; Abbott, A. P., et al., ChemPhysChem 7 (2006) 803) or by a freeze-drying procedure (Gutierrez, M. C., et al., Langmuir 25 (2009) 5509; ibid Angew. Chem. Int. Ed. 49 (2010) 2158).

For example, choline chloride is mixed with an aliphatic hydrogen bond donor comprising at least one thiol group and at least one hydroxy group or salt thereof (e.g., dithiothreitol, 2-mercaptoethanol, 4-mercapto-1-butanol, 1-mercapto-2-propanol or sodium 2-mercaptoethansulfonate) at elevated temperature (e.g. at 120° C.) in the required molar ratio with agitation for half an hour and cooling the heated combination afterwards (after the DES has been formed). To remove traces of water drying with phosphor pentoxide can be performed afterwards (e.g. for two weeks at 45° C.).

Also for example, choline chloride and the aliphatic hydrogen bond donor comprising at least one thiol group and at least one hydrogen group or salt thereof (dithiothreitol, 2-mercaptoethanol, 4-mercapto-1-butanol, 1-mercapto-2-propanol or sodium 2-mercaptoethansulfonate) are mixed at the intended ratio (i.e. 1:22 to 1:3 or 1:1, respectively) and mixed, using e.g. a magnetic stirrer, over a flame. After a colorless clear liquid had formed the mixture is cooled down to room temperature. Thereafter drying over phosphor pentoxide in a desiccator is possible (e.g. at room temperature, 2 weeks).

Another exemplary method is reported in U.S. Pat. No. 8,247,198: nitrogen salt (0.05 mol) and hydrogen bond donor (0.1 mol for choline chloride mixtures, 0.075 mol for ethyl ammonium chloride mixtures) were added to a 20-ml vial and heated at 80° C., until a clear, homogenous liquid formed, typically one hour.

According to U.S. Pat. No. 8,247,198 the components within the DES's are surprisingly and significantly 20 to more than 600-fold less reactive for enzymes than expected. It is reported therein a method comprising enzymatic catalysis of a chemical reaction in a solution comprising a deep eutectic solvent. The reaction may be, e.g., transesterification, aminolysis, hydrolysis, perhydrolysis, and/or alcohol dehydrogenase activity. The reaction may be a polymerization reaction. The polymerization reaction may be catalyzed by an enzyme that is, e.g., a member of the group consisting of enzymes that catalyze transesterification, aminolysis, hydrolysis, perhydrolysis, alcohol dehydrogenation, oxidation-reduction, or dehydrogenation. The reaction may produce, e.g., an addition product or a condensation product. The polymerization reaction may produce, e.g., a polyester or a polyamide. An enzyme that catalyzes the enzyme-catalyzed chemical reaction may be, e.g., a member of the group consisting of transesterase, hydrolase, lipase, amidase, and dehydrogenase (column 2, lines 4 to 19).

Deep eutectic solvents can comprise organic or aqueous components as co-solvents. For example, in one embodiment, the deep eutectic solvent as reported herein comprises between 10% and 99% (v/v) of deep eutectic solvent and the remainder is a non-deep eutectic solvent.

In one embodiment the hydrogen bond acceptor is selected from the group consisting of organic acids, urea, thiourea, amide, hydroxyl groups, diols, glycerol, choline chloride, and combinations thereof. In one embodiment the hydrogen bond acceptor is selected from the group comprising choline chloride, ethyl ammonium chloride, choline bromide, glycerol, tetrabutyl ammonium chloride, triethyl benzyl ammonium chloride, zinc chloride, and acetylcholine chloride. In one preferred embodiment the deep eutectic solvent comprises as first component choline chloride and as second component dithiothreitol or sodium 2-mercaptoethansulfonate.

In one embodiment the nitrogen salt as hydrogen bond acceptor is selected from compounds with a positively charged nitrogen atom. In one embodiment the nitrogen salt is selected from the group consisting of primary, secondary, tertiary, and quaternary nitrogen components. In one embodiment the nitrogen salt is an ammonium salt or a substituted ammonium compound. In one embodiment the nitrogen salt is a halide-nitrogen salt. In one embodiment the metal salt is a transition metal salt. In one embodiment the metal salt is a halide-metal salt. In one embodiment the hydrogen bond donor is selected from the group consisting of hydroxyl, amide, amine, aldehyde, and carboxylic acid. Transition metals are the elements from the period table of the chemical elements that have a number of 21 to 30, 39 to 48, 71 to 80, and 103 to 112. Halides are fluoride (F—), chloride (Cl—), bromide (Br—), iodide (I—) and astatide (At—). In one embodiment the components of the DES have a specific ratio based on their used volume. In one embodiment the ratio is from about 1:3 parts to about 3:1 parts of nitrogen or metal salt to hydrogen bond donor comprising at least one thiol group.

When used in an enzymatic reaction also other components will be present in the DES, such as e.g. an enzyme and its substrates. Due to the fact that some enzyme are not available in solid form (either conjugated to a solid support or as powder) a DES used in an enzymatic reaction will comprises to a certain percentage a co-solvent, in most cases water or an aqueous buffer. Thus, the DES accounts for at least 95%, at least 90%, at least 85%, or at least 80% of the volume of the enzymatic reaction mixture and the rest is made up of water or an aqueous buffer. In one preferred embodiment the enzymatic reaction mixture comprises at least 90% of DES, more preferably about 95% of DES.

Many reactions can be performed in DES, e.g. comprising an enzyme and a DES and optionally a co-solvent. The enzyme and its substrates are present in effective concentrations and at ratios useful to make the intended product. An effective concentration does not include concentrations that are not suitable to make the intended product in meaningful quantities and in a reasonable time. In one embodiment the enzyme is used/is present in an amount/concentration of at least about 0.1, at least about 1, at least about 5, at least about 10, or at least about 20 mg enzyme per ml of solvent, i.e. from about 0.1 to about 10 mg/ml. Similarly, the enzymatic substrates are in one embodiment present in a concentration of at least about 1%, at least about 5%, at least about 10%, at least about 20%, or at least about 30% wt substrate/wt of solvent, i.e. from about 1% to about 25%.

### III. Enzymatic Conjugation Using Sortase A

A covalent conjugate (i.e. a fusion polypeptide) comprising two in vivo not covalently associated entities can be obtained in vitro by using the enzyme sortase, especially a Sortase A.

Transamidases in general catalyze the formation of a peptide bond (amide bond) between an acyl donor and a nucleophilic acyl acceptor. In order to form a peptide bond the acyl acceptor has to contain a NH2—CH2-moiety. Gram-positive bacteria include the following genera: Actinomyces, Bacillus, Bifidobacterium, Cellulomonas, Clostridium, Corynebacterium, Micrococcus, Mycobacterium, Nocardia, Staphylococcus, Streptococcus and Streptomyces.

Sortases have been classified into 4 classes, designated A, B, C, and D, based on sequence alignment and phylogenetic analysis of 61 sortases from gram-positive bacterial genomes (Dramsi, S., et al., Res. Microbiol. 156 (2005) 289-297). These classes correspond to the following subfamilies, into which sortases have also been classified by Comfort and Clubb (Comfort, D. and Clubb, R. T., Infect. Immun. 72 (2004) 2710-2722): Class A (Subfamily 1), Class B (Subfamily 2), Class C (Subfamily 3), Class D (Subfamilies 4 and 5). The aforementioned references disclose numerous sortases and recognition motifs (see also Pallen, M. J., et al., Trends Microbiol. 9 (2001) 97-101). With this information a person skilled in the art can assign a sortase to the correct class based on its sequence and/or other characteristics such as those described in Dramsi (supra).

Sortase A (SrtA) is a membrane bound enzyme has transamidase activity. It has been identified and isolated from gram-positive bacteria. In vivo Sortase A attaches proteins covalently to the bacterial cell wall. The specific recognition motif on the SrtA substrate is LPXTG (SEQ ID NO: 01), whereby the enzyme cleaves between the residues threonine and glycine. The recognition motif on the peptidoglycan is a pentaglycine motif. It has been shown that a triglycine and even a diglycine motif on the N-terminus is sufficient to support the SrtA reaction (Clancy, K. W., et al., Peptide Science 94 (2010) 385-396). The reaction proceeds through a thioester acyl-enzyme intermediate, which is resolved by the attack of an amine nucleophile from the oligoglycine, covalently linking peptidoglycan to a protein substrate and regenerating SrtA. SrtA can be used to covalently conjugate chemically synthetized peptides to recombinantly expressed proteins.

Many gram-positive bacteria use sortase to covalently anchor a variety of surface proteins including virulence factors to their cell wall (peptidoglycan). Sortases are membrane associated enzymes. The wild-type Staphylococcus aureus sortase A (SrtA) is a polypeptide of 206 amino acids with an N-terminal membrane-spanning region. In a first step, sortase A recognizes substrate proteins that contain a LPXTG (SEQ ID NO: 01) amino acid sequence motif and cleaves the amide bond between the Thr and Gly by means of an active-site Cys. This peptide cleaving reaction results in a sortase A-substrate thioester intermediate. In a second step the thioester acyl-enzyme intermediate is resolved by nucleophilic attack of an amino group of an oligoglycine containing second substrate polypeptide (corresponding to the pentaglycine unit of peptidoglycan in S. aureus) leading to a covalently linked cell wall protein and the regeneration of sortase A. In the absence of oligoglycine nucleophiles, the acyl-enzyme intermediate can be hydrolyzed by a water molecule.

Sortase-mediated ligation/conjugation has begun to be applied for a variety of protein engineering and bioconjugation purposes. This technique enables the introduction of natural and synthetic functionalities into LPXTG-tagged recombinant or chemically synthesized polypeptides. Examples include the covalent attachment of oligoglycine derivatized polymers (e.g. PEG), fluorophores, vitamins (e.g. biotin and folate), lipids, carbohydrates, nucleic acids, synthetic peptides and proteins (e.g. GFP) (see e.g. Tsukiji, S. and Nagamune, T., ChemBioChem 10 (2009) 787-798; Popp, M. W. L. and Ploegh, H. L., Angew. Chem. Int. Ed. Engl. 50 (2011) 5024-5032).

For the enzymatic conjugation a soluble truncated sortase A lacking the membrane-spanning region (SrtA; amino acid residues 60-206 of Staphylococcus aureus SrtA) can be used (SEQ ID NO: 05; see also Ton-That, H., et al., Proc. Natl. Acad. Sci. USA 96 (1999) 12424-12429; Ilangovan, H., et al., Proc. Natl. Acad. Sci. USA 98 (2001) 6056-6061).

The sortase A-mediated reaction results in the ligation of species containing a sortase motif (sequence) with those bearing one or more N-terminal glycine residues. The sortase motif can be the amino acid sequence LPXTG (SEQ ID NO:

01), but can also different therefrom (see below). However, a drawback of using such sequences as acyl donors is that the transfer of the LPXT (SEQ ID NO: 35) unit to a nucleophilic acyl acceptor liberates a stoichiometric amount of a corresponding fragment containing at least one N-terminal glycine residue. The liberated glycine-containing fragment competes with the intended acyl acceptor for the enzymatic intermediate and works against the progress of the enzymatic ligation reaction. Additionally the hydrolytic cleavage of the enzymatic intermediate as well as the LPXTG containing substrate, although a relatively slow process, competes with the reaction. In the beginning of the use of the sortase-mediated reaction useful levels of ligation could only be obtained using concentrations of at least 5 mM of the acyl donor comprising the sortase-motif.

The general sortase-motif has the amino acid sequence LPXT, wherein X can be any amino acid residue, i.e. a naturally occurring amino acid residue or a non-naturally occurring amino acid residue. In some embodiments, X is selected from the group of amino acid residues comprising or consisting of (in one letter code) D, E, A, N, Q, K, and R. In some embodiments, the sortase-motif is selected from the group comprising or consisting of the amino acid sequences LPXT, LPXA, SPXT, LAXT, LSXT, NPXT, VPXT, IPXT, LGXT, and YPXR (SEQ ID NO: 35, 70-78). In some embodiments, the sortase motif is selected from the group of amino acid sequences consisting of LPST, LPKT, LPIT, LPDT, SPKT, LAET, LAAT, LAST, LPLT, LSRT, LPET, VPDT, IPQT, YPRR, LPMT, LAFT, and LPQT (SEQ ID NO: 79-95). In certain embodiments in which sortase A is used, the sortase-motif comprises the amino acid sequence X1PX2X3 (SEQ ID NO: 96), wherein i) X1 is selected from the group consisting of the amino acid residues leucine, isoleucine, valine and methionine, ii) X2 is any amino acid, and iii) X3 is selected from the group consisting of threonine, serine and alanine. In specific embodiments, as noted above X1, is leucine and X3 is threonine. In certain embodiments X2 is selected from the group consisting of aspartate, glutamate, alanine, glutamine, lysine and methionine.

In some embodiments the sortase-motif is selected from the group of amino acid sequences comprising or consisting of LPKTG, LPITG, LPDTA, SPKTG, LAETG, LAATG, LAHTG, LASTG, LPLTG, LSRTG, LPETG, VPDTG, IPQTG, YPRRG, LPMTG, LAFTG, and LPQTS (SEQ ID NO: 38, 44, 45, 46, 47, 48, 49, 50, 51, 52, 35, 53, 54, 97, 43, 98, 58). In some embodiments of the invention the sortase is a sortase A (SrtA). SrtA recognizes a sortase-motif with the amino acid sequence LPXTG (SEQ ID NO: 01). Common sortase-motif amino acid sequences are, e.g., LPKTG, LPATG, LPETG and LPNTG (SEQ ID NO: 38, 65, 04, and 39). In some embodiments LPETG (SEQ ID NO: 04) is used. However, sortase-motifs not in line with this consensus sortase-motif amino acid sequence may also be recognized. For example, in some embodiments the sortase-motif comprises the amino acid residue A rather than the amino acid residue T at position 4, e.g. LPXAG or LPNAG (SEQ ID NO: 99, 100). In some embodiments the sortase-motif comprises the amino acid residue A rather than the amino acid residue G at position 5, e.g. LPXTA or LPNTA (SEQ ID NO: 101, 102). In some embodiments the sortase-motif comprises the amino acid residue G rather than the amino acid residue P at position 2, e.g. LGXTG or LGATG. In some embodiments the sortase-motif comprises the amino acid residue I rather than the amino acid residue L at position 1, e.g., IPXTG or IPNTG or IPETG (SEQ ID NO: 105, 106, 107).

In some embodiments, where the sortase-motif is LPXTG or LPXT (SEQ ID NO: 01, 35), X is selected from the group consisting of D, E, A, N, Q, K, and R. In some embodiments X is selected from the group of amino acid residues consisting of K, E, N, Q, and A in an LPXTG or LPXT motif where the sortase is a sortase A. In one embodiment the sortase-motif is LPET or LPETG or LPETA (SEQ ID NO: 89, 04, and 108).

In certain embodiments where sortase A from Staphylococcus aureus (St.au. SrtA) is used the sortase-motif has the amino acid sequence LPX1TX2 (SEQ ID NO: 109), wherein i) X1 is selected from the group of amino acid residues consisting of D, E, A, N, Q, K, and R, and ii) X2 is selected from the group of amino acid residues consisting of alanine and glycine. In certain embodiments the sortase-motif of St.au. SrtA is LPX1TA (SEQ ID NO: 110). In other embodiments the sortase-motif of St.au. SrtA is LPX1TG (SEQ ID NO: 111). X1 has the meaning as outlined before.

Streptococcus pyogenes sortase A (St.py. SrtA) will accept di-alanine based nucleophiles. This sortase will efficiently cleave the sortase-motif amino acid sequence LPXTA (SEQ ID NO: 101) between the threonine and the alanine residue and install modified alanine-based nucleophiles. St.py. SrtA will also recognize and cleave LPXTG (SEQ ID NO: 01) motifs, albeit with reduced efficiency.

Staphylococcus aureus sortase A (St.au. SrtA) will not significantly cleave LPXTA (SEQ ID NO: 101) motifs or accept alanine based nucleophiles.

In one embodiment, a polypeptide is contacted with Strep. SrtA and an alanine-containing nucleophile. The polypeptide comprises a sortase-motif amino acid sequence that can be recognized by Strep. SrtA at or near its C-terminus and the nucleophile comprises one or more amino acids capable of serving as nucleophile for a St.au. SrtA-mediated reaction at or near its N-terminus (e.g., (G)n, where n is between 1 and 10, e.g., between 1 and 5). This leads to the formation of an LPXTA (SEQ ID NO: 101) sequence at the reactive site, a motif refractory to cleavage by St.au. SrtA. This allows for example St.au. SrtA to act on the N-terminus without affecting the C-terminal modification installed with Strep. SrtA.

Sortase fragments having sortase transamidation activity can be used in the methods as reported herein. Sortase fragments can be identified by producing fragments of sortase, for example, by recombinant techniques or proteolytic digestion of full length sortase, and determining the rate of peptide bond formation, i.e. ligation. The fragment can comprise about 80% of amino acid sequence of full-length sortase, about 70%, about 60%, about 50%, about 40% or about 30% of the amino acid sequence of full-length sortase such as that of S. aureus Sortase A (GenBank Accession number AAD48437). In some embodiments the fragment lacks an N-terminal portion of the full-length sortase amino acid sequence that is not essential to the catalytic activity of sortase, for example the fragment lacks the N-terminal portion extending to the end of the membrane anchor sequence. In some embodiments the fragment comprises the C-terminus of a full-length sortase amino acid sequence. In some embodiments, the fragment comprises the catalytic core region of a sortase. In one embodiment the core region is from about position 60 to about position 206 of SrtA, e.g., S. aureus SrtA, or about from position 82 to about position 249 of Strep. SrtA.

Sortases from other organisms also can be utilized in the processes as reported herein. Such sortases often are encoded by nucleotide sequences substantially identical or similar to the nucleotide sequences that encode SrtA. A similar or substantially identical nucleotide sequence may include modifications to the native sequence, such as substitutions, deletions, or insertions of one or more nucleotides. Included are nucleotide sequences that are at least 55%, 60%, 65%, 70%, 75%, 80%, or 85% or more identical to a native nucleotide sequence, and often are 90% or 95% or more identical to the native nucleotide sequence (each identity percentage can include a 1%, 2%, 3% or 4% variance). One test for determining whether two nucleic acids are substantially identical is to determine the percentage of identical nucleotide positions shared between two nucleic acids.

SrtA nucleotide sequences may be used as “query sequences” to perform a search against public databases to identify related sequences. Such searches can be performed using the NBLAST and)(BLAST programs (version 2.0) of Altschul, et al. (J. Mol. Biol. 215 (1990) 403-410). BLAST nucleotide searches can be performed with the NBLAST program, score =100, word-length=12 to obtain homologous nucleotide sequences. To obtain gapped alignments for comparison purposes, gapped BLAST can be utilized as described in Altschul, et al. (Nuc. Acids Res. 25 (1997) 3389-3402). When utilizing BLAST and Gapped BLAST programs, default parameters of the respective programs (e.g., XBLAST and NBLAST) can be used (see e.g. www.ncbi.nlm.nih.gov).

A variant amino acid sequence departs from a native amino acid sequence. Amino acid substitutions may be made on the basis of similarity in polarity, charge, solubility, hydrophobicity, hydrophilicity, helix-forming properties and/or amphipathic properties and the resulting variants are screened for enzymatic activity with a suitable assay, such as that reported in European patent application EP14198535. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine. In certain embodiments, conservative substitutions may be made, according to the following Table. Amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

| | | |
|---|---|---|
| aliphatic amino acid | non-polar | G, A, P |
| residues | | I, L, V |
| | polar, non-charged | C, S, T, M |
| | | N, Q |
| | polar, charged | D, E |
| | | K, R |
| aromatic | | H, F, W, Y |

In certain embodiments homologous substitution may occur, which is a substitution or replacement of like amino acids, such as basic for basic, acidic for acidic, polar for polar amino acids, and hydrophobic for hydrophobic, for example. Non-homologous substitutions can be introduced to a native sequence, such as from one class of residue to another (e. g. a non-hydrophobic to a hydrophobic amino acid), or substituting a naturally occurring amino acid with an unnatural amino acids or non-classical amino acid replacements.

In the methods as reported herein the sortase, the sortase-motif comprising polypeptide (i.e. the acyl donor), and the nucleophile (i.e. the acyl acceptor) are incubated together under conditions suitable to effect the formation of a peptide bond between the N-terminal part of the sortase-motif comprising polypeptide and the nucleophile. As used herein, the term “incubating” or grammatical equivalents thereof denotes that the components of the process are brought in close proximity to one another to allow contact between the molecules. Incubating can be done by adding them to one reaction vessel, for example. The components in the system may be mixed in a variety of manners, such as by oscillating a vessel, subjecting a vessel to a vortex generating apparatus, or repeated mixing with a pipette or pipettes, for example. The components may be added in any order to the system.

The sortase reaction may be performed in any convenient vessel (e.g., tubes such as microfuge tubes, flask, dish), microtiter plates (e.g., 96-well or 384-well plates), glass slides, silicon chips, filters, or any solid or semisolid support having surface (optionally coated) having molecules immobilized thereon and optionally oriented in an array (see, e.g., US 6,261,776 and Fodor, Nature 364 (1993) 555-556), and microfluidic devices (see, e.g., U.S. Pat. No. 6,440,722; U.S. Pat. No. 6,429,025; U.S. Pat. No. 6,379,974; and U.S. Pat. No. 6,316,781).

The reaction mixture is generally cell free and further does not include bacterial cell wall components or intact bacterial cell walls. In some embodiments, the sortase-motif comprising polypeptide and/or the nucleophile are expressed by one or more recombinant nucleotide sequences in a cell, which nucleotide sequences are integrated into the cell genome or non-integrated (e.g., in a plasmid).

The reaction mixture is maintained at any convenient temperature at which the sortase reaction can be performed. In some embodiments, the sortase reaction is performed at a temperature between and including about 15° C. and about 50° C. In some embodiments, the sortase reaction is performed at a temperature between and including about 23° C. and about 37° C. In certain embodiments, the temperature is room temperature (i.e. about 20° C. to 25° C.). The temperature can be optimized by repetitively performing the same sortase reaction at different temperatures and determining ligation rates.

Any convenient volume and component ratio can be used.

In certain embodiments, a (molar) ratio of 1:1000 or greater of sortase enzyme to sortase-motif comprising polypeptide is utilized, or a (molar) ratio of 1:1000 or greater of sortase enzyme to nucleophile is utilized. In specific embodiments, ratios of sortase enzyme to sortase-motif comprising polypeptide or enzyme to nucleophile is about 1:1, including 1:2 or greater, 1:3 or greater, 1:4 or greater, 1:5 or greater, 1:6 or greater, 1:7 or greater, 1:8 or greater, and 1:9 or greater.

In some embodiments, the sortase-motif comprising polypeptide is present at a concentration ranging from about 10 μM to about 10 mM. In some embodiments, the sortase-motif comprising polypeptide is present at a concentration ranging from about 100 μM to about 1 mM. In some embodiments, the sortase-motif comprising polypeptide is present at a concentration ranging from about 100 μM to about 50 mM. In some embodiments, the sortase-motif comprising polypeptide is present at a concentration ranging from about 200 μM to about 1 mM. In some embodiments, the sortase-motif comprising polypeptide is present at a concentration ranging from about 200 μM to about 800 μM. In some embodiments, the sortase-motif comprising polypeptide is present at a concentration ranging from about 400 μM to about 600 μM.

In certain embodiments the nucleophile is present in excess with respect to the sortase-motif comprising polypeptide. In certain embodiments, the nucleophile is present in 10-fold excess with respect to the sortase-motif polypeptide. In certain embodiments, the nucleophile is present in 25-fold excess with respect to the sortase-motif polypeptide. In certain embodiments, the nucleophile is present in 50-fold excess with respect to the sortase-motif polypeptide. In certain embodiments, the nucleophile is present in 100-fold excess with respect to the sortase-motif polypeptide. In certain embodiments, the nucleophile is present in 250-fold excess with respect to the sortase-motif polypeptide.

In certain embodiments, the nucleophile is present at a concentration ranging from about 1 μM to about 50 mM. In certain embodiments, the nucleophile is present at a concentration ranging from about 15 μM to about 1500 μM. In certain embodiments, the nucleophile is present at a concentration ranging from about 25 μM to about 1000 μM. In certain embodiments, the nucleophile is present at a concentration ranging from about 40 μM to about 250 μM.

In certain embodiments, the sortase is present at a concentration ranging from about 1 μM to about 500 μM. In certain embodiments, the sortase is present at a concentration ranging from about 15 μM to about 150 μM. In certain embodiments, the sortase is present at a concentration ranging from about 25 μM to about 100 μM. In certain embodiments, the sortase is present at a concentration ranging from about 40 μM to about 60 μM.

In certain embodiments, the method is performed in a reaction mixture comprising an aqueous environment. Water with an appropriate buffer and/or salt content often may be utilized. An alcohol or organic solvent may be included in certain embodiments. The amount of an organic solvent often does not appreciably esterify a protein or peptide in the ligation process (e.g., esterified protein or peptide often increase only by 5% or less upon addition of an alcohol or organic solvent). Alcohol and/or organic solvent contents sometimes are 20% or less, 15% or less, 10% or less or 5% or less, and in embodiments where a greater amount of an alcohol or organic solvent is utilized, 30% or less, 40% or less, 50% or less, 60% or less, 70% or less, or 80% or less alcohol or organic solvent is present. In certain embodiments, the reaction mixture includes only an alcohol or an organic solvent, with only limited amounts of water if it is present.

In some embodiments, the reaction mixture comprises a buffer. A person skilled in the art will be familiar with a variety of buffers that could be used in accordance with the methods as reported herein. In some embodiments, the buffer solution comprises calcium ions. In certain embodiments, the buffer solution does not contain substances that precipitate calcium ions. In some embodiments, the buffer solution does not include phosphate ions. In some embodiments, the buffer solution does not contain chelating agents.

In some embodiments, the method is performed at a pH value in the range of from 6 to 8.5. In some embodiments, the method is performed at a pH value in the range of from 6 to 8. In some embodiments, the method is performed at a pH value in the range of from 6 to 7.5. In some embodiments, the method is performed at a pH value in the range of from 6.5 to 8.5. In some embodiments, the method is performed at a pH value in the range of from 7 to 8.5. In some embodiments, the method is performed at a pH value in the range of from 7.5 to 8.5. In some embodiments, the method is performed at a pH value in the range of from 7.0 to 8.5. In some embodiments, the method is performed at a pH value in the range of from 7.3 to 7.8.

One or more components of the reaction mixture or the product may be immobilized to a solid support. The attachment between the reaction mixture component and the solid support may be covalent or non-covalent (see, e.g., U.S. Pat. No. 6,022,688 for non-covalent attachments). The solid support may be one or more surfaces of the system, such as one or more surfaces in each well of a microtiter plate, a surface of a glass slide or silicon wafer, BIAcore chip, a surface of a particle, e.g., a bead (see e.g., Lam, Nature 354 (1991) 82-84) that is optionally linked to another solid support, or a channel in a microfluidic device, for example.

Types of solid supports, linker molecules for covalent and non-covalent attachments to solid supports, and methods for immobilizing molecules to solid supports are known (see, e.g., U.S. Pat. No. 6,261,776; U.S. Pat. No. 5,900,481; U.S. Pat. No. 6,133,436; U.S. Pat. No. 6,022,688; WO 2001/18234). Any material may be used, e.g., plastic (e.g., polystyrene), metal, glass, cellulose, gels (e.g., formed at least in part from organic polymers such as PDMS), etc. In some embodiments the solid support is semi-solid and/or gel-like, deformable, flexible, or the like.

Any polypeptide, eventually after introduction of a sortase-motif or an oligoglycine or -alanine, may be used as sortase-motif comprising polypeptide or nucleophile in the methods as reported herein.

Summarizing the above, the first substrate, also denoted as donor, comprises the sortase recognition motif. It is cleaved by the sortase after the threonine residue in the recognition motif. Thereby a C-terminal activated carboxyl group (acyl intermediate) is generated. The second substrate, also denoted as acceptor or nucleophile, provides a (free N-terminal) amino group. Between the free amino group and the activated carboxyl group a peptide bond is formed in the sortase catalyzed transpeptidation reaction.

Thus, for the enzymatic sortase mediated transpeptidation reaction it is only required that a donor comprising a sortase recognition motif and an acceptor comprising an N-terminal free glycine, alanine, cysteine or an equivalent functional group is incubated with a polypeptide having sortase A catalytic activity. The remainder of the donor as well as of the acceptor does not interfere with the reaction.

Thus, a sortase mediated transpeptidation reaction can be performed with virtually any protein or small molecule independently of each other as donor or acceptor as long as these comprise a pair of sortase recognition sequence and nucleophile.

This is confirmed by the art.

For example, Marraffini et al. (Microbiol. Mol. Biol. Rev. 70 (2006) 192-221) reported that sortase A can be used to incorporate chemicals containing glycine residues with a free amino group to the LPXTG motif of recombinant proteins, i.e. without limitation of the protein. Presented examples are the conjugation of triglycyl-lysine-folate with (GFP or Cre or p27)-LPETG-His6 with high efficiency, the incorporation of the branched peptide AT-P-022 into polypeptides, and the self-cleavage of chimeras of His6-sortase-LPETG-target protein (the fusion cleaves itself once the enzyme has been activated by the addition of calcium and triglycine).

Further, Antos et al. (J. Am. Chem. Soc. 131 (2009) 10800-10801) reported that the transpeptidation reaction catalyzed by sortase A allows site-specific derivatization of proteins with virtually any type of functional material. Target proteins are engineered to contain the recognition site (LPXTG) near their C terminus, thus allowing a trans-acylation reaction in which the residues C-terminal to threonine are exchanged for a synthetic oligoglycine peptide. It is reported that the terminal G residue of the sortase recognition motif can be replaced by a methyl ester without imparting the reaction. In this document nucleophiles comprising either a fluorescent label or a protein were used for the conjugation to cholera toxin B subunit.

Further, Popp et al. (Proc. Natl. Acad. Sci. USA 108 (2011) 3169-3174) reported the use of Sortase for polypeptide cyclization and PEGylation. The method is general and applicable to a wide variety of proteins. The sortase transpeptidase reaction allows facile site-specific PEGylation of multiple distinct proteins, as exemplified using interferon a2, GCSF, and erythropoietin. In all cases tested, the site-specific C-terminal PEGylation proceeded efficiently.

In EP 2 990 423 a self-cleaving sortase construct is reported. In this construct the sortase recognition sequence LPETG and the catalytic sortase domain have been combined in the same molecule. As protein comprising the sortase recognition sequence any protein, such as e.g. a protein selected from the group comprising polymer proteins, glycoproteins, cytokines, growth factor, blood preparations, vaccines, hormones, enzymes, antibodies and parts or fragments thereof (isolated light or heavy chains).

### IV. Sortases

Full length Streptococcus pyogenes Sortase A (Uniprot Q1J6K9; catalytic core underlined; conserved histidine underlined) has the following amino acid sequence:

(SEQ ID NO: 159)
MVKKQKRRKI KSMSWARKLL IAVLLILGLA LLFNKPIRNT
LIARNSNKYQ VTKVSKKQIK KNKEAKSTFD FQAVEPVSTE
SVLQAQMAAQ QLPVIGGIAI PELGINLPIF KGLGNTELIY
GAGTMKEEQV MGGENNYSLA SHHIFGITGS SQMLFSPLER
AQNGMSIYLT DKEKIYEYII KDVFTVAPER VDVIDDTAGL
KEVTLVTCTD IEATERIIVK GELKTEYDFD KAPADVLKAF
NHSYNQVST.

The amino acid sequence of the mature soluble sortase A derived from Streptococcus pyogenes is

(SEQ ID NO: 156)
VLQAQMAAQQ LPVIGGIAIP ELGINLPIFK GLGNTELIYG
AGTMKEEQVM GGENNYSLAS HHIFGITGSS QMLFSPLERA
QNGMSIYLTD KEKIYEYIIK DVFTVAPERV DVIDDTAGLK
EVTLVTCTDI EATERIIVKG ELKTEYDFDK APADVLKAFN
HSYNQVST.

Full length Staphylococcus aureus Sortase A (see Mazmanian et al.; catalytic core underlined; conserved histidine underlined) has the following amino acid sequence:

(SEQ ID NO: 26)
MKKWTNRLMT IAGVVLILVA AYLFAKPHID NYLHDKDKDE
KIEQYDKNVK EQASKDKKQQ AKPQIPKDKS KVAGYIEIPD
ADIKEPVYPG PATPEQLNRG VSFAEENESL DDQNISIAGH
TFIDRPNYQF TNLKAAKKGS MVYFKVGNET RKYKMTSIRD
VKPTDVGVLD EQKGKDKQLT LITCDDYNEK TGVWEKRKIF
VATEVK.

Staphylococcus aureus Sortase A without the N-terminal 28 amino acid residues (N(2-29) transmembrane domain) has the following amino acid sequence:

(SEQ ID NO: 160)
MDNYLHDKDK DEKIEQYDKN VKEQASKDKK QQAKPQIPKD
KSKVAGYIEI PDADIKEPVY PGPATPEQLN RGVSFAEENE
SLDDQNISIA GHTFIDRPNY QFTNLKAAKK GSMVYFKVGN
ETRKYKMTSI RDVKPTDVGV LDEQKGKDKQ LTLITCDDYN
EKTGVWEKRK IFVATEVK.

Staphylococcus aureus Sortase A without the N-terminal 59 amino acid residues (transmembrane domain) has the following amino acid sequence:

(SEQ ID NO: 05)
QAKPQIPKDK SKVAGYIEIP DADIKEPVYP GPATPEQLNR
GVSFAEENES LDDQNISIAG HTFIDRPNYQ FTNLKAAKKG
SMVYFKVGNE TRKYKMTSIR DVKPTDVGVL DEQKGKDKQL
TLITCDDYNE KTGVWEKRKI FVATEVK

Full length Sortase A from Listeria monocytogenes has the following amino acid sequence (the catalytic center is underlined; the conserved histidine is underlined):

(SEQ ID NO: 161)
MLKKTIAAAA LAAGLLLIFS PFIKNGIVKY MSGHETIEQY
KASDIKKNNE KDATFDFESV QLPSMTSVIK GAANYDKDAV
VGSIAVPSVD VNLLVFKGTN TANLLAGATT MRSDQVMGKG
NYPLAGHHMR DESMLFGPIM KVKKGDKIYL TDLENLYEYT
VTETKTIDET EVSVIDDTKD ARITLITCDK PTETTKRFVA
VGELEKTEKL TKELENKYFP SK.

The amino acid sequence of the mature soluble sortase A derived from Listeria monocytogenes Sortase A has the following amino acid sequence:

(SEQ ID NO: 06)
ANYDKDAVVG SIAVPSVDVN LLVFKGTNTA NLLAGATTMR
SDQVMGKGNY PLAGHHMRDE SMLFGPIMKV KKGDKIYLTD
LENLYEYTVT ETKTIDETEV SVIDDTKDAR ITLITCDKPT
ETTKRFVAVG ELEKTEKLTK ELENKYFPSK

### V. The Method and Use as Reported Herein

One aspect as reported herein is a method for the enzymatic production of a polypeptide comprising the following step

incubating
i) a first polypeptide (optionally comprising within the 100 C-terminal amino acid residues) the amino acid sequence LPXTG (SEQ ID NO: 01, wherein X can be any amino acid residue) or LPXTA (SEQ ID NO: 101, wherein X can be any amino acid residue),
ii) a second polypeptide that has i) a glycinyl, an alaninyl, or a cysteinyl compound at its N-terminus, or ii) an oligoglycine, or oligoalanine, or a cysteine amino acid residue followed by one to three glycine or alanine amino acid residues at its N-terminus, or iii) a lysine amino acid residue within its 5 N-terminal amino acid residues, and
iii) a third polypeptide with sortase A activity,

in a deep eutectic solvent as reported herein

and thereby producing a polypeptide.

In one embodiment the third polypeptide with sortase A activity is derived from Staphylococcus aureus sortase A, or from Streptococcus pyogenes Sortase A, or from Listeria monocytogenes Sortase A. In one embodiment the third polypeptide comprises the amino acid sequence of SEQ ID NO: 05, SEQ ID NO: 06, SEQ ID NO: 27, SEQ ID NO: 156, SEQ ID NO: 159 or SEQ ID NO: 161. In one preferred embodiment the third polypeptide comprises the amino acid sequence of SEQ ID NO: 05 or 06.

In one embodiment the third polypeptide comprises additionally a tag at its N- or C-terminus either conjugated directly or via an intervening linker. In one embodiment the third polypeptide is consisting of the amino acid sequence of SEQ ID NO: 05 and the C-terminal tag of SEQ ID NO: 32. In one embodiment the third polypeptide is consisting of the amino acid sequence of SEQ ID NO: 05.

In one embodiment the method is for the enzymatic conjugation of two polypeptides.

In one embodiment the deep eutectic solvent comprises choline chloride. In one embodiment the deep eutectic solvent is a mixture of choline chloride with dithiothreitol at a molar ratio of 1:2.5 to 1:3 or with sodium 2-mercaptoethansulfonate at a molar ratio of 1:1. In one embodiment the deep eutectic solvent comprises an aqueous co-solvent. In one embodiment the deep eutectic solvent comprises more than 0% and up to about 10% (v/v) co-solvent. In one embodiment the deep eutectic solvent comprises more than 0% and up to about 5% (v/v) co-solvent. In one embodiment the deep eutectic solvent comprises of from about 1%, 2%, 3%, or 4% and up to about 6%, 7%, 8%, 9%, or 10% (v/v) aqueous co-solvent. Any combination of the lower value and the upper value is an embodiment herein. In one preferred embodiment the deep eutectic solvent is a mixture of choline chloride with dithiothreitol glycerol at a molar ratio of 1:2.5 to 1:3 or with sodium 2-mercaptoethansulfonate at a molar ratio of 1:1 comprising more than 0% and up to about 5% (v/v) aqueous co-solvent.

In one embodiment the second polypeptide has at its N-terminus the amino acid sequence GGG, AAA, CGG, CAA, KGG, or KAA (SEQ ID NO: 29, 162-166).

In one embodiment the first polypeptide comprises (optionally within the 250 C-terminal amino acid residues) the amino acid sequence LPXTG (SEQ ID NO: 01, wherein X can be any amino acid residue) or LPXTA (SEQ ID NO: 101, wherein X can be any amino acid residue). In one embodiment the first polypeptide comprises (optionally within the 100 C-terminal amino acid residues) the amino acid sequence LPXTG (SEQ ID NO: 01, wherein X can be any amino acid residue) or LPXTA (SEQ ID NO: 101, wherein X can be any amino acid residue). In one embodiment the first polypeptide comprises within the 25 C-terminal amino acid residues the amino acid sequence LPXTG (SEQ ID NO: 01, wherein X can be any amino acid residue) or LPXTA (SEQ ID NO: 101, wherein X can be any amino acid residue). In one embodiment the first polypeptide comprises within the 10 C-terminal amino acid residues the amino acid sequence LPXTG (SEQ ID NO: 01, wherein X can be any amino acid residue) or LPXTA (SEQ ID NO: 101, wherein X can be any amino acid residue).

In one embodiment the first polypeptide comprises at its C-terminus the amino acid sequence LPXTG (SEQ ID NO: 01, wherein X can be any amino acid residue) or LPXTA (SEQ ID NO: 101, wherein X can be any amino acid residue). In one embodiment the first polypeptide comprises at its C-terminus the amino acid sequence LPETG (SEQ ID NO: 04) or LPETA (SEQ ID NO: 108).

In one embodiment the first polypeptide and the second polypeptide are independently of each other selected from an antibody variable domain, an antibody heavy chain Fab-fragment, an antibody Fc-region, a tag, and a peptide comprising the amino acid sequence LPXTG (SEQ ID NO: 01, wherein X can be any amino acid residue) or LPXTA (SEQ ID NO: 101, wherein X can be any amino acid residue), a linker and a non-sortase motif moiety.

One aspect as reported herein is the use of a deep eutectic solvent comprising choline chloride and dithiothreitol at a molar ratio of 1:2.5 to 1:3 or sodium 2-mercaptoethansulfonate at a molar ratio of 1:1 and further comprising up to about 5% (v/v) aqueous co-solvent as solvent in an enzymatic transamidation reaction catalyzed by Sortase A.

One aspect as reported herein is a method for the Sortase A catalyzed production of a polypeptide comprising the following step

incubating in a thiol-based deep eutectic solvent
i) a first polypeptide comprising (optionally within the 20 C-terminal amino acid residues) the amino acid sequence LPXTG (SEQ ID NO: 01, wherein X can be any amino acid residue) or LPXTA (SEQ ID NO: 101, wherein X can be any amino acid residue),
ii) a second polypeptide that has i) a glycinyl, an alaninyl, or a cysteinyl compound at its N-terminus, or ii) an oligoglycine, or oligoalanine, or a cysteine amino acid residue followed by one to three glycine or alanine amino acid residues at its N-terminus, or iii) a lysine amino acid residue within its 5 N-terminal amino acid, and
iii) a soluble sortase that has the amino acid sequence of SEQ ID NO: 05, SEQ ID NO: 06, SEQ ID NO: 27, SEQ ID NO: 156, SEQ ID NO: 159 or SEQ ID NO: 161 and optionally comprising the C-terminal tag of SEQ ID NO: 32,

whereby the thiol-based deep eutectic solvent comprises
a) choline chloride and dithiothreitol at a molar ratio of from 1:2.5 to 1:3, or
b) choline chloride and 2-mercaptoethanol at a molar ratio of about 1:2, or
c) choline chloride and 4-mercapto-1-butanol at a molar ratio of about 1:2, or
d) choline chloride and 1-mercapto-2-propanol at a molar ratio of about 1:2, or
e) choline chloride and sodium 2-mercaptoethansulfonate at a molar ratio of about 1:1,
and
more than 0% and up to about 10% (v/v) aqueous co-solvent and thereby producing a polypeptide.

The First or Second Polypeptide

The sortase-motif (amino acid sequence) may be conjugated to or incorporated in, if it is not directly comprises in one of these molecules, a therapeutic agent (drug), a cytotoxic agent (e.g. a toxin such as doxorubicin or pertussis toxin), a fluorophores such as a fluorescent dye like fluorescein or rhodamine, a chelating agent for an imaging or radiotherapeutic metal, a peptidyl or non-peptidyl label, a tag, or a clearance-modifying agent such as various isomers of polyethylene glycol, a peptide that binds to a third component, another carbohydrate or lipophilic agent, or a small molecule, such as e.g. a synthetic small molecule (e.g. acetyl salicylic acid). If the motif is incorporated via conjugation the conjugation can be either directly or via an intervening linker. Furthermore the first and/or second polypeptide can either be recombinantly produced or can be synthetic or semi-synthetic, i.e. recombinantly produced and thereafter chemically modified.

a) Therapeutic Agents

The therapeutic agent can be any compound, moiety or group which has a therapeutic effect, such as e.g. an antibody, a cytotoxic or cytostatic compound. The antibody can be a full length or complete antibody or an antigen-binding fragment thereof.

A number of therapeutic antibodies directed against cell surface molecules and their ligands are known, such as Rituxan/MabThera/Rituximab, 2H7/Ocrelizumab, Zevalin/Ibrizumomab, Arzerra/Ofatumumab (CD20), HLL2/Epratuzumab, Inotuzomab (CD22), Zenapax/Daclizumab, Simulect/Basiliximab (CD25), Herceptin/Trastuzumab, Pertuzumab (Her2/ERBB2), Mylotarg/Gemtuzumab (CD33), Raptiva/Efalizumab (C dl 1 a), Erb itux/C etuximab (EGFR, epidermal growth factor receptor), IMC-1121B (VEGF receptor 2), Tysabri/Natalizumab (a4-subunit of α4β1 and α4β7 integrins), ReoPro/Abciximab (gpIIb-gpIIa and αvβ3-integrin), Orthoclone OKT3/Muromonab-CD3 (CD3), Benlysta/Belimumab (BAFF), Tolerx/Oteliximab (CD3), Soliris/Eculizumab (C5 complement protein), Actemra/Tocilizumab (IL-6R), Panorex/Edrecolomab (EpCAM, epithelial cell adhesion molecule), CEA-CAMS/Labetuzumab (CD66/CEA, carcinoembryonic antigen), CT-11 (PD-1, programmed death-1 T-cell inhibitory receptor, CD-d279), H224G11 (c-Met receptor), SAR3419 (CD19), IMC-A12/Cixutumumab (IGF -1R, insulin-like growth factor 1 receptor), MEDI-575 (PDGF-R, platelet-derived growth factor receptor), CP-675, 206/Tremelimumab (cytotoxic T lymphocyte antigen 4), R05323441 (placenta growth factor or PGF), HGS1012/Mapatumumab (TRAIL-R1), SGN-70 (CD70), Vedotin(SGN-35)/Brentuximab (CD30), and ARH460-16-2 (CD44).

The conjugates obtained with the method as reported herein can be used in the preparation of medicaments for the treatment of e.g. an oncologic disease, a cardiovascular disease, an infectious disease, an inflammatory disease, an autoimmune disease, a metabolic (e.g., endocrine) disease, or a neurological (e.g. neurodegenerative) disease. Exemplary non-limiting examples of these diseases are Alzheimer's disease, non-Hodgkin's lymphomas, B-cell acute and chronic lymphoid leukemias, Burkitt lymphoma, Hodgkin's lymphoma, hairy cell leukemia, acute and chronic myeloid leukemias, T-cell lymphomas and leukemias, multiple myeloma, glioma, Waldenstrom's macroglobulinemia, carcinomas (such as carcinomas of the oral cavity, gastrointestinal tract, colon, stomach, pulmonary tract, lung, breast, ovary, prostate, uterus, endometrium, cervix, urinary bladder, pancreas, bone, liver, gall bladder, kidney, skin, and testes), melanomas, sarcomas, gliomas, and skin cancers, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcal nephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitis obliterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, pernicious anemia, rapidly progressive glomerulonephriti s, psoriasis, or fibrosing alveolitis.

A number of cell surface markers and their ligands are known. For example cancer cells have been reported to express at least one of the following cell surface markers and or ligands, including but not limited to, carbonic anhydrase IX, alpha fetoprotein, alpha-actinin-4, A3 (antigen specific for A33 antibody), ART-4, B7, Ba-733, BAGE, BrE3-antigen, CA125, CAMEL, CAP-1, CASP-8/m, CCCL19, CCCL21, CD1, CD1a, CD2, CD3, CD4, CDS, CD8, CD1-1A, CD14, CD15, CD16, CD18, CD19, CD20, CD21, CD22, CD23, CD25, CD29, CD30, CD32b, CD33, CD37, CD38, CD40, CD40L, CD45, CD46, CD54, CD55, CD59, CD64, CD66a-e, CD67, CD70, CD74, CD79a, CD80, CD83, CD95, CD126, CD133, CD138, CD147, CD154, CDC27, CDK-4/m, CDKN2A, CXCR4, CXCR7, CXCL12, HIF-1-alpha, colon-specific antigen-p (CSAp), CEA (CEACAM5), CEACAM6, c-met, DAM, EGFR, EGFRvIII, EGP-1, EGP-2, ELF2-M, Ep-CAM, Flt-1, Flt-3, folate receptor, G250 antigen, GAGE, GROB, HLA-DR, HM1.24, human chorionic gonadotropin (HCG) and its subunits, HER2/neu, HMGB-1, hypoxia inducible factor (HIF-1), HSP70-2M, HST-2or 1a, IGF-1R, IFN-gamma, IFN-alpha, IFN-beta, IL-2, IL-4R, IL-6R, IL-13R, IL-15R, IL-17R, IL-18R, IL-6, IL-8, IL-12, IL-15, IL-17, IL-18, IL- 25, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUCS, MUM-1/2, MUM-3, NCA66, NCA95, NCA90, pancreatic cancer mucin, placental growth factor, p53, PLAGL2, prostatic acid phosphatase, PSA, PRAME, PSMA, P1GF, ILGF, ILGF-1R, IL-6, IL-25, RSS, RANTES, T101, SAGE, S100, survivin, survivin-2B, TAC, TAG-72, tenascin, TRAIL receptors, TNF-alpha, Tn-antigen, Thomson-Friedenreich antigens, tumor necrosis antigens, VEGFR, ED-B fibronectin, WT-1, 17-1A-antigen, complement factors C3, C3a, C3b, C5a, C5, an angiogenesis marker, bcl-2, bcl-6, Kras, cMET, an oncogene marker and an oncogene product (see, e.g., Sensi, et al., Clin. Cancer Res. 12 (2006) 5023-5032; Parmiani, et al, J. Immunol. 178 (2007) 1975-1979; Novellino, et al., Cancer Immunol. Immunother. 54 (2005) 187-207).

Thus, antibodies recognizing specific cell surface receptors including their ligands can be used for specific and selective targeting and binding to a number/multitude of cell surface markers that are associated with a disease. A cell surface marker is a polypeptide located on the surface of a cell (e.g. a disease-related cell) that is e.g.

associated with signaling event or ligand binding.

In one embodiment, for the treatment of cancer/tumors multispecific binding molecules/bispecific antibodies are produced that target tumor-associated antigens, such as those reported in Herberman, “Immunodiagnosis of Cancer”, in Fleisher (ed.), “The Clinical Biochemistry of Cancer”, page 347 (American Association of Clinical Chemists (1979)) and in U.S. Pat. No. 4,150,149; U.S. Pat. No. 4,361,544; and U.S. Pat. No. 4,444,744.

Reports on tumor associated antigens (TAAs) include Mizukami, et al., (Nature Med. 11 (2005) 992-997); Hatfield, et al., (Curr. Cancer Drug Targets 5 (2005) 229-248); Vallbohmer, et al., (J Clin. Oncol. 23 (2005) 3536-3544); and Ren, et al., (Ann. Surg. 242 (2005) 55-63), each incorporated herein by reference with respect to the TAAs identified.

Where the disease involves a lymphoma, leukemia or autoimmune disorder, targeted antigens may be selected from the group consisting of CD4, CDS, CD8, CD14, CD15, CD19, CD20, CD21, CD22, CD23, CD25, CD33, CD37, CD38, CD40, CD40L, CD46, CD54, CD67, CD74, CD79a, CD80, CD126, CD138, CD154, CXCR4, B7, MUC1 or la, HM1.24, HLA-DR, tenascin, VEGF, P1GF, ED-B fibronectin, an oncogene, an oncogene product (e.g., c-met or PLAGL2), CD66a-d, necrosis antigens, IL-2, T101, TAG, IL-6, MIF, TRAIL-R1 (DR4) and TRAIL-R2 (DRS).

A number of bispecific antibodies are known directed against two different targets , such as BCMA/CD3, different antigens of the HER family in combination (EGFR, HER2, HER3), CD19/CD3, IL17RA/IL7R, IL-6/IL-23, IL-1-beta/IL-8, IL-6 or IL 6R/ IL-21 or IL-21R, first specificity directed to a glycoepitope of an antigen selected from the group consisting of Lewis x-, Lewis b- and Lewis y-structures, Globo H-structures, KH1, Tn-antigen, TF-antigen and carbohydrate structures of Mucins, CD44, glycolipids and glycosphingolipids, such as Gg3, Gb3, GD3, GD2, Gb5, Gm1, Gm2, sialyltetraosylceramide and a second specificity directed to an ErbB receptor tyrosine kinase selected from the group consisting of EGFR, HER2, HER3 and HER4, GD2 in combination with a second antigen binding site is associated with an immunological cell chosen from the group consisting of T lymphocytes NK cell, B-lymphocytes, dendritic cells, monocytes, macrophages, neutrophils, mesenchymal stem cells, neural stem cells, ANG2/VEGF, VEGF/PDGFR-beta, Vascular Endothelial Growth Factor (VEGF) acceptor 2/CD3, PSMA/CD3, EPCAM/CD3, combinations of an antigen is selected from a group consisting of VEGFR-1, VEGFR-2, VEGFR-3, FLT3, c FMS/CSF1R, RET, c-Met, EGFR, Her2/neu, HER3, HER4, IGFR, PDGFR, c-KIT, BCR, integrin and MMPs with a water-soluble ligand is selected from the group consisting of VEGF, EGF, PIGF, PDGF, HGF, and angiopoietin, ERBB-3/C-MET, ERBB-2/C-MET, EGF receptor 1/CD3, EGFR/HER3, PSCA/CD3, C-MET/CD3, ENDOSIALIN/CD3, EPCAM/CD3, IGF-1R/CD3, FAPALPHA/CD3, EGFR/IGF-1R, IL 17A/F, EGF receptor 1/CD3, and CD19/CD16.

Toxic drug moieties include: (i) chemotherapeutic agents, which may function as microtubule inhibitors, mitosis inhibitors, topoisomerase inhibitors, or DNA intercalators; (ii) protein toxins, which may function enzymatically; and (iii) radioisotopes.

Exemplary toxic drug moieties include, but are not limited to, a maytansinoid, an auristatin, a dolastatin, a trichothecene, CC1065, a calicheamicin and other enediyne antibiotics, a taxane, an anthracycline, and stereoisomers, isosters, analogs or derivatives thereof.

Protein toxins include diphtheria-A chain, non-binding active fragments of diphtheria toxin, exotoxin A chain (from Pseudomonas aeruginosa), ricin A chain (Vitetta et al (1987) Science, 238:1098), abrin A chain, modeccin A chain, alpha-sarcin, Aleurites fordii proteins, dianthin proteins, Phytolaca americana proteins (PAPI, PAPII, and PAP -5), momordica charantia inhibitor, curcin, crotin, sapaonaria officinalis inhibitor, gelonin, mitogellin, restrictocin, phenomycin, enomycin, and the tricothecenes (WO 93/21232).

Therapeutic radioisotopes include 32P, 33P, 90Y, 125I, 131I, 131In, 153 Sm, 186Re, 188Re, 211At, 212B, 212Pb, and radioactive isotopes of Lu.

The radioisotope or other labels may be incorporated in known ways (Fraker et al (1978) Biochem. Biophys. Res. Commun. 80: 49-57; “Monoclonal Antibodies in Immunoscintigraphy” Chatal, CRC Press 1989). Carbon-14-labeled 1-isothiocyanatobenzyl-3-methyldiethylene triamine pentaacetic acid (MX-DTPA) is an exemplary chelating agent for conjugation of a radionuclide to the complex (WO 94/11026).

b) Labels

The non-sortase motif moiety can be a label. Any label moiety which can be covalently attached to the sortase amino acid sequence can be used (see e.g. Singh et al (2002) Anal. Biochem. 304:147-15; Harlow E. and Lane, D. (1999) Using Antibodies: A Laboratory Manual, Cold Springs Harbor Laboratory Press, Cold Spring Harbor, N.Y.; Lundblad R. L. (1991) Chemical Reagents for Protein Modification, 2nd ed. CRC Press, Boca Raton, Fla.). The label may function to: (i) provide a detectable signal; (ii) interact with a second label to modify the detectable signal provided by the first or second label, e.g. to give FRET (fluorescence resonance energy transfer); (iii) affect mobility, e.g. electrophoretic mobility or cell-permeability, by charge, hydrophobicity, shape, or other physical parameters, or (iv) provide a capture moiety, e.g. to modulate ionic complexation.

Conjugates comprising a haptenylated label as reported herein may be useful in diagnostic assays, e.g., for detecting expression of an antigen of interest in specific cells, tissues, or serum. For diagnostic applications, a bispecific antibody will be used wherein the first binding specificity binds to a target and the second binding specificity binds to a haptenylated label. The hapten will typically be labeled with a detectable moiety. Numerous labels are available which can be generally grouped into the following categories:

(a) Radioisotopes (radionuclides), such as 3H, 11C, 14C, 18F, 32P, 35S, 64Cu, 68Gn, 86Y, 89Zr, 99TC, 111In, 123I, 124I, 125I, 131I, 133Xe, 177Lu, 211At, or 131Bi. Radioisotope labeled conjugates are useful in receptor targeted imaging experiments. The antigen (hapten) can be labeled with ligand reagents that bind, chelate or otherwise complex a radioisotope metal using the techniques described in Current Protocols in Immunology, (1991) Volumes 1 and 2, Coligen et al, Ed. Wiley-Interscience, New York, N.Y., Pubs. Chelating ligands which may complex a metal ion include DOTA, DOTP, DOTMA, DTPA and TETA (Macrocyclics, Dallas, Tex.). Radionuclides can be targeted via complexation with the complex as reported herein (Wu et al, Nature Biotechnology 23(9) (2005) 1137-1146). Receptor target imaging with radionuclide labeled complexes can provide a marker of pathway activation by detection and quantification of progressive accumulation of complexes or corresponding therapeutic antibodies in tumor tissue (Albert et al (1998) Bioorg. Med. Chem. Lett. 8:1207-1210).

Metal-chelate complexes suitable as labels for imaging experiments (US 2010/0111856; U.S. Pat. No. 5,342,606; U.S. Pat. No. 5,428,155; U.S. Pat. No. 5,316,757; U.S. Pat. No. 5,480,990; U.S. Pat. No. 5,462,725; U.S. Pat. No. 5,428,139; U.S. Pat. No. 5,385,893; U.S. Pat. No. 5,739,294; U.S. Pat. No. 5,750,660; U.S. Pat. No. 5,834,456; Hnatowich et al, J. Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990), Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, et al, J. Nucl. Med. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals 18 (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

(b) Fluorescent labels such as rare earth chelates (europium chelates), fluorescein types including FITC, 5-carboxyfluorescein, 6-carboxy fluorescein; rhodamine types including TAN/IRA; dansyl; Lissamine; cyanines; phycoerythrins; Texas Red; and analogs thereof. The fluorescent labels can be conjugated to the antigen (hapten) using the techniques disclosed in Current Protocols in Immunology, supra, for example. Fluorescent dyes and fluorescent label reagents include those which are commercially available from Invitrogen/Molecular Probes (Eugene, Oregon, USA) and Pierce Biotechnology, Inc. (Rockford, Ill.).

Detection labels such as fluorescent dyes and chemiluminescent dyes (Briggs et al “Synthesis of Functionalised Fluorescent Dyes and Their Coupling to Amines and Amino Acids,” J. Chem. Soc., Perkin-Trans. 1 (1997) 1051-1058) provide a detectable signal and are generally applicable for labeling, especially with the following properties: (i) the labeled conjugate should produce a very high signal with low background so that small quantities of conjugate can be sensitively detected in both cell-free and cell-based assays; and (ii) the labeled conjugate should be photostable so that the fluorescent signal may be observed, monitored and recorded without significant photo bleaching. For applications involving cell surface binding of labeled conjugates to membranes or cell surfaces, especially live cells, the labels should (iii) have good water-solubility to achieve effective conjugate concentration and detection sensitivity and (iv) are non-toxic to living cells so as not to disrupt the normal metabolic processes of the cells or cause premature cell death.

(c) Various enzyme-substrate labels are available or disclosed (see e.g. U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP), (3-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to polypeptides are described in O'Sullivan et al “Methods for the Preparation of Enzyme-Antibody Conjugates for use in Enzyme Immunoassay”, in Methods in Enzym. (ed. by J. Langone & IT Van Vunakis), Academic Press, New York, 73 (1981) 147-166.

Examples of enzyme-substrate combinations (U.S. Pat. No. 4,275,149; U.S. Pat. No. 4,318,980) include, for example:

(i) Horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB));

(ii) alkaline phosphatase (AP) with para-nitrophenyl phosphate as chromogenic substrate; and

(iii) (3-D-galactosidase ((3-D-Gal) with a chromogenic substrate (e.g., p-nitro phenyl-(3-D-galactosidase) or fluorogenic substrate 4-methylumb elliferyl-(3-D-galactosidase.

The labeled conjugate as reported herein may be employed in any known assay method, such as ELISA, competitive binding assays, direct and indirect sandwich assays, and immunoprecipitation assays (Zola, Monoclonal Antibodies: A Manual of Techniques (1987) pp. 147-158, CRC Press, Inc.).

Labeled conjugates as reported herein are useful as imaging biomarkers and probes by the various methods and techniques of biomedical and molecular imaging such as: (i) MRI (magnetic resonance imaging); (ii) MicroCT (computerized tomography); (iii) SPECT (single photon emission computed tomography); (iv) PET (positron emission tomography) Tinianow, J. et al, Nuclear Medicine and Biology, 37(3) (2010) 289-297; Chen et al, Bioconjugate Chem. 15 (2004) 41-49; US 2010/0111856 (v) bioluminescence; (vi) fluorescence; and (vii) ultrasound. Immunoscintigraphy is an imaging procedure in which conjugates labeled with radioactive substances are administered to an animal or human patient and a picture is taken of sites in the body where the conjugate localizes (U.S. Pat. No. 6,528,624). Imaging biomarkers may be objectively measured and evaluated as an indicator of normal biological processes, pathogenic processes, or pharmacological responses to a therapeutic intervention. Biomarkers may be of several types: Type 0 markers are natural history markers of a disease and correlate longitudinally with known clinical indices, e.g. MRI assessment of synovial inflammation in rheumatoid arthritis; Type I markers capture the effect of an intervention in accordance with a mechanism-of-action, even though the mechanism may not be associated with clinical outcome; Type II markers function as surrogate endpoints where the change in, or signal from, the biomarker predicts a clinical benefit to “validate” the targeted response, such as measured bone erosion in rheumatoid arthritis by CT. Imaging biomarkers thus can provide pharmacodynamic (PD) therapeutic information about: (i) expression of a target protein, (ii) binding of a therapeutic to the target protein, i.e. selectivity, and (iii) clearance and half-life pharmacokinetic data. Advantages of in vivo imaging biomarkers relative to lab-based biomarkers include: non-invasive treatment, quantifiable, whole body assessment, repetitive dosing and assessment, i.e. multiple time points, and potentially transferable effects from preclinical (small animal) to clinical (human) results. For some applications, bioimaging supplants or minimizes the number of animal experiments in preclinical studies.

Peptide labeling methods are well known. See Haugland (2003) Molecular Probes Handbook of Fluorescent Probes and Research Chemicals, Molecular Probes, Inc.; Brinkley (1992) Bioconjugate Chem. 3:2; Garman, (1997) Non-Radioactive Labeling: A Practical Approach, Academic Press, London; Means (1990) Bioconjugate Chem. 1:2; Glazer et al Chemical Modification of Proteins.

Laboratory Techniques in Biochemistry and Molecular Biology (T. S. Work and E. Work, Eds.) American Elsevier Publishing Co., New York; Lundblad, R. L. and Noyes, C. M. (1984) Chemical Reagents for Protein Modification, Vols. I and II, CRC Press, New York; Pfleiderer, G. (1985) “Chemical Modification of Proteins”, Modern Methods in Protein Chemistry, H. Tschesche, Ed., Walter DeGruyter, Berlin and New York; and Wong (1991) Chemistry of Protein Conjugation and Cross-linking, CRC Press, Boca Raton, Fla.); DeLeon-Rodriguez et al, Chem. Eur. J. 10 (2004) 1149-1155; Lewis et al, Bioconjugate Chem. 12 (2001) 320-324; Li et al, Bioconjugate Chem. 13 (2002) 110-115; Mier et al Bioconjugate Chem. 16 (2005) 240-237.

c) Linker

The term “linker” denotes a bifunctional or multifunctional moiety which can be used to conjugate (link) a first moiety with a second moiety. Linked conjugates can be conveniently prepared using a linker having two reactive functionalities.

In one embodiment, a linker has a reactive site which has an electrophilic group that is reactive to a nucleophilic group present in the sortase amino acid sequence. Useful electrophilic groups include, but are not limited to, another thiol, maleimide and haloacetamide groups (see e.g. conjugation method at page 766 of Klussman et al, Bioconjugate Chemistry 15(4) (2004) 765-773).

Examples of thiol-reaction functional groups include, but are not limited to, thiol, maleimide, alpha-haloacetyl, activated esters such as succinimide esters, 4-nitrophenyl esters, pentafluorophenyl esters, tetrafluorophenyl esters, anhydrides, acid chlorides, sulfonyl chlorides, isocyanates and isothiocyanates.

The linker may comprise amino acid residues which link the sortase amino acid sequence to the non-sortase motif moiety. The amino acid residues may form a dipeptide, tripeptide, tetrapeptide, pentapeptide, hexapeptide, heptapeptide, octapeptide, nonapeptide, decapeptide, undecapeptide or dodecapeptide unit. Amino acid residues include those occurring naturally, as well as non-naturally occurring amino acid analogs, such as e.g. citrulline or β-amino acids, such as e.g. β-alanine, or ω-amino acids such as 4-amino-butyric acid.

In another embodiment, the linker has a reactive functional group which has a nucleophilic group that is reactive to an electrophilic group present in the sortase amino acid sequence. Useful electrophilic groups include, but are not limited to, aldehyde and ketone carbonyl groups. The heteroatom of a nucleophilic group of a linker can react with an electrophilic group in the sortase amino acid sequence and form a covalent bond to the sortase amino acid sequence. Useful nucleophilic groups on a linker include, but are not limited to, hydrazide, oxime, amino, hydrazine, thiosemicarbazone, hydrazine carboxylate, and arylhydrazide. The electrophilic group on an antigen (hapten) provides a convenient site for attachment to a linker.

Typically, peptide-type linkers can be prepared by forming a peptide bond between two or more amino acids and/or peptide fragments. Such peptide bonds can be prepared, for example, according to the liquid phase synthesis method (E. Schroder and K. Lubke “The Peptides”, volume 1 (1965) 76-136, Academic Press) which is well known in the field of peptide chemistry.

In another embodiment, the linker may be substituted with groups which modulated solubility or reactivity. For example, a charged substituent such as sulfonate (SO3—) or ammonium or a polymer such as PEG, may increase water solubility of the reagent and facilitate the coupling reaction of the linker reagent with the antigen (hapten) or the drug moiety, or facilitate the coupling reaction depending on the synthetic route employed.

The conjugates comprising a non-sortase motif moiety as reported herein expressly contemplate, but are not limited to, complexes prepared with linker reagents: BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone) benzoate), and including bis-maleimide reagents: DTME, BMB, BMDB, BMH, BMOE, BM(PEO)3, and BM(PEO)4, which are commercially available from Pierce Biotechnology, Inc. Bis-maleimide reagents allow the attachment of e.g. a thiol group to a thiol-containing drug moiety, label, or linker intermediate, in a sequential or concurrent fashion. Other functional groups besides maleimide, which are reactive with e.g. a thiol group, include iodoacetamide, bromoacetamide, vinyl pyridine, disulfide, pyridyl disulfide, isocyanate, and isothiocyanate.

Exemplary linker include a valine-citrulline (val-cit or vc) dipeptide linker reagent having a maleimide stretcher and a para-aminobenzylcarbamoyl (PAB) self-immolative spacer, and a phe-lys(Mtr) dipeptide linker reagent having a maleimide Stretcher unit and a p-amino benzyl self-immolative spacer.

Cysteine thiol groups are nucleophilic and capable of reacting to form covalent bonds with electrophilic groups on linker reagents and the non-sortase motif moiety or the sortase amino acid sequence including: (i) active esters such as NHS esters, HOBt esters, haloformates, and acid halides; (ii) alkyl and benzyl halides, such as haloacetamides; (iii) aldehydes, ketones, carboxyl, and maleimide groups; and (iv) disulfides, including pyridyl disulfides, via sulfide exchange. Nucleophilic groups on a haptenylated compound include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents.

### V. Recombinant Methods

Any polypeptide domain (e.g. a single chain antigen binding polypeptide such as a scFv, a scFab, or a darpin, or a multi chain antigen binding polypeptide such as a dsFv or a Fab) comprising an nucleophilic amino acid sequence at its N-terminus, such as e.g. an oligoglycine motif (GG (SEQ ID NO: 28), GGG (SEQ ID NO: 29), GGGG (SEQ ID NO: 30), GGGGG (SEQ ID NO: 31)) can be expressed and purified from the supernatant of eukaryotic cells (e.g. HEK293 cells, CHO cells). It does not matter if the polypeptide is an isolated polypeptide or comprised in a multimeric or heteromeric entity.

Suitable host cells for cloning or expression/secretion of polypeptide-encoding vectors include prokaryotic or eukaryotic cells described herein. For example, polypeptides may be produced in bacteria, in particular when glycosylation is not needed (see, e.g., U.S. Pat. No. 5,648,237, U.S. Pat. No. 5,789,199 and U.S. Pat. No. 5,840,523, Charlton, Methods in Molecular Biology 248 (2003) 245-254 (B.K.C. Lo, (ed.), Humana Press, Totowa, N.J.), describing expression of antibody fragments in E. coli). After expression, the polypeptide may be isolated from the bacterial cell paste in a soluble fraction or may be isolated from the insoluble fraction so called inclusion bodies which can be solubilized and refolded to bioactive forms. Thereafter the polypeptide can be further purified.

In addition to prokaryotes, eukaryotic microbes such as filamentous fungi or yeasts are suitable cloning or expression hosts for polypeptide-encoding vectors, including fungi and yeast strains whose glycosylation pathways have been “humanized”, resulting in the production of a polypeptide with a partially or fully human glycosylation pattern (see e.g. Gerngross, Nat. Biotech. 22 (2004) 1409-1414, and Li, et al., Nat. Biotech. 24 (2006) 210-215).

Suitable host cells for the expression of glycosylated polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts (see, e.g., U.S. Pat. No. 5,959,177, U.S. Pat. No. 6,040,498, U.S. Pat. No. 6,420,548, U.S. Pat. No. 7,125,978 and U.S. Pat. No. 6,417,429 (describing PLANTIBODIES™ technology for producing antibodies in transgenic plants)).

Vertebrate cells may also be used as hosts. For example, mammalian cell lines that are adapted to grow in suspension may be useful. Other examples of useful mammalian host cell lines are the COS-7 cell line (monkey kidney CV1 cell transformed by SV40); the HEK293 cell line (human embryonic kidney); the BHK cell line (baby hamster kidney); the TM4 mouse sertoli cell line (TM4 cells as described, e.g., in Mather, Biol. Reprod. 23 (1980) 243-251); the CV1 cell line (monkey kidney cell); the VERO-76 cell line (African green monkey kidney cell);

the HELA cell line (human cervical carcinoma cell); the MDCK cell line (canine kidney cell); the BRL-3A cell line (buffalo rat liver cell); the W138 cell line (human lung cell); the HepG2 cell line (human liver cell); the MMT 060562 cell line (mouse mammary tumor cell); the TRI cell line (e.g. described in Mather, et al., Anal. N.Y. Acad. Sci. 383 (1982) 44-68); the MRCS cell line; and the FS4 cells-line. Other useful mammalian host cell lines include the CHO cell line (Chinese hamster ovary cell), including DHFR negative CHO cell lines (see e.g. Urlaub, et al., Proc. Natl. Acad. Sci. USA 77 (1980) 4216), and myeloma cell lines such as Y0, NS0 and Sp2/0 cell line. For a review of certain mammalian host cell lines suitable for polypeptide production, see, e.g., Yazaki, and Wu, Methods in Molecular Biology, Antibody Engineering 248 (2004) 255-268 (B.K.C. Lo, (ed.), Humana Press, Totowa, NJ).

### DESCRIPTION OF THE FIGURES

FIG. 1 Chromatogram of the reaction product of Example 4; 8.433 min=GGG-biotin, 13.350 min=Sa-SrtA, 20.942=LCR640-LPETG (educt), 21.987 min=LCR640-LPETG-biotin (product).

The following examples, figures and sequences are provided to aid the understanding of the present invention, the true scope of which is set forth in the appended claims. It is understood that modifications can be made in the procedures set forth without departing from the spirit of the invention.

### EXAMPLES

### Recombinant DNA Techniques

Standard methods were used to manipulate DNA as described in Sambrook, J. et al., Molecular cloning: A laboratory manual; Cold Spring Harbor Laboratory Press, Cold Spring Harbor, N.Y., 1989. The molecular biological reagents were used according to the manufacturer's instructions.

### Gene and Oligonucleotide Synthesis

Desired gene segments were prepared by chemical synthesis at Geneart GmbH (Regensburg, Germany). The synthesized gene fragments were cloned into an E. coli plasmid for propagation/amplification. The DNA sequences of subcloned gene fragments were verified by DNA sequencing. Alternatively, short synthetic DNA fragments were assembled by annealing chemically synthesized oligonucleotides or via PCR. The respective oligonucleotides were prepared by metabion GmbH (Planegg-Martinsried, Germany).

### Description of the Basic/Standard Mammalian Expression Plasmid

For the expression of a desired gene/protein (e.g. full length antibody heavy chain, full length antibody light chain, or an Fc-chain containing an oligoglycine at its N-terminus) a transcription unit comprising the following functional elements is used:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a gene/protein to be expressed (e.g. shortened Sortase A of Staphylococcus aureus), and
the bovine growth hormone polyadenylation sequence (BGH pA).

Beside the expression unit/cassette including the desired gene to be expressed the basic/standard mammalian expression plasmid contains
an origin of replication from the vector pUC18 which allows replication of this plasmid in E. coli, and
a beta-lactamase gene which confers ampicillin resistance in E. coli.

### Protein Determination

The protein concentration of purified polypeptides was determined by determining the optical density (OD) at 280 nm, using the molar extinction coefficient calculated on the basis of the amino acid sequence of the polypeptide.

### Example 1

Generation of an Expression Plasmid for Soluble Sortase A

### Staphylococcus aureus Derived Sortase A

The sortase gene encodes an N-terminally truncated Staphylococcus aureus sortase A (60-206) molecule (amino acid sequence of SEQ ID NO: 05).

The expression plasmid for the transient expression of soluble sortase in HEK293 cells comprised besides the soluble sortase expression cassette an origin of replication from the vector pUC18, which allows replication of this plasmid in E. coli, and a beta-lactamase gene which confers ampicillin resistance in E. coli.̂

The transcription unit of the soluble sortase comprised the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a purification tag encoding nucleic acid,
an N-terminally truncated S. aureus sortase A encoding nucleic acid, and
the bovine growth hormone polyadenylation sequence (BGH pA).

The amino acid sequence of the mature soluble sortase is

(SEQ ID NO: 05)
QAKPQIPKDKSKVAGYIEIPDADIKEPVYPGPATPEQLNRGVSFAEENES
LDDQNISIAGHTFIDRPNYQFTNLKAAKKGSMVYFKVGNETRKYKMTSIR
DVKPTDVGVLDEQKGKDKQLTLITCDDYNEKTGVWEKRKIFVATEVK.

The purification tag has the amino acid sequence MRGSHHHHHHGS (SEQ ID NO: 32).

### Staphylococcus pyogenes Derived Sortase A

The sortase gene encodes an N-terminally truncated Staphylococcus pyogenes sortase A molecule (amino acid sequence of SEQ ID NO: 156).

The expression plasmid for the transient expression of soluble sortase in HEK293 cells comprised besides the soluble sortase expression cassette an origin of replication from the vector pUC18, which allows replication of this plasmid in E. coli, and a beta-lactamase gene which confers ampicillin resistance in E. coli. ̂

The transcription unit of the soluble sortase comprised the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a purification tag encoding nucleic acid,
an N-terminally truncated S. pyogenes sortase A encoding nucleic acid, and
the bovine growth hormone polyadenylation sequence (BGH pA).

The amino acid sequence of the mature soluble sortase is

(SEQ ID NO: 156)
VLQAQMAAQQLPVIGGIAIPELGINLPIFKGLGNTELIYGAGTMKEEQVM
GGENNYSLASHHIFGITGSSQMLFSPLERAQNGMSIYLTDKEKIYEYIIK
DVFTVAPERVDVIDDTAGLKEVTLVTCTDIEATERIIVKGELKTEYDFDK
APADVLKAFNHSYNQVST.

The purification tag has the amino acid sequence MRGSHHHHHHGS (SEQ ID NO: 32).

### Listeria monocytogenes Derived Sortase A

The sortase gene encodes an N-terminally truncated Listeria monocytogenes sortase A (73-222) molecule (amino acid sequence of SEQ ID NO: 06).

The expression plasmid for the transient expression of soluble sortase in HEK293 cells comprised besides the soluble sortase expression cassette an origin of replication from the vector pUC18, which allows replication of this plasmid in E. coli, and a beta-lactamase gene which confers ampicillin resistance in E. coli.

The transcription unit of the soluble sortase comprised the following functional elements:
the immediate early enhancer and promoter from the human cytomegalovirus (P-CMV) including intron A,
a human heavy chain immunoglobulin 5′-untranslated region (5′UTR),
a murine immunoglobulin heavy chain signal sequence,
a purification tag encoding nucleic acid,
an N-terminally truncated L. monocytogenes sortase A encoding nucleic acid, and
the bovine growth hormone polyadenylation sequence (BGH pA).

The purification tag has the amino acid sequence MRGSHHHHHHGS (SEQ ID NO: 32).

### Example 2

Transient Expression and Analytical Characterization

The recombinant production was performed by transient transfection of HEK293 cells (human embryonic kidney cell line 293-derived) cultivated in F17 Medium (Invitrogen Corp.). For transfection “293-Fectin” Transfection Reagent (Invitrogen) was used. Transfection was performed as specified in the manufacturer's instructions. Cell culture supernatants were harvested three to seven (3-7) days after transfection. Supernatants were stored at reduced temperature (e.g. −80° C.).

General information regarding the recombinant expression of human immunoglobulins in e.g. HEK293 cells is given in: Meissner, P. et al., Biotechnol. Bioeng. 75 (2001) 197-203.

The protein concentration was determined by measuring the optical density (OD) at 280 nm, using the molar extinction coefficient calculated on the basis of the amino acid sequence. Purity was analyzed by SDS-PAGE in the presence and absence of a reducing agent (5 mM 1,4-dithiotreitol) and staining with Coomassie brilliant blue.

### Example 3

Production of Thiol-based Deep Eutectic Solvents

### Dithiothreitol and Choline Chloride

Choline chloride and dithiothreitol were mixed in a 1:2.5 molar ratio. Choline chloride (0.1 mol) was employed as a dry powder and were added to 0.25 mol of dithiothreitol. The mixture was heated slowly over a flame and shaken until a clear, uniform solution had been formed.

The liquid was allowed to cool to room temperature. The yield was quantitative and the product had a melting point of lower than room temperature. About 10 g of the synthesized deep eutectic solvent were put in a closed vial and kept in a dry space.

### Beta-Mercaptoethanol and Choline Chloride

Choline chloride and beta-mercaptoethanol were mixed in a 1:2 molar ratio. Choline chloride (0.1 mol) was employed as a dry powder and was added to 0.2 mol of beta-mercaptoethanol. The mixture comprising was heated slowly over a flame and shaken until a uniform and clear solution had been formed.

The liquid was allowed to cool to room temperature. The yield was quantitative and the product had a melting point of lower than room temperature. About 10 g of the synthesized deep eutectic solvent were put in a closed vial and kept in a dry space.

### 4-Mercapto-1-Butanol and Choline Chloride

Choline chloride and 4-mercapto-1-butanol were mixed in a 1:2 molar ratio. Choline chloride (0.1 mol) was employed as a thy powder and was added to 0.2 mol of 4-mercapto-1-butanol. The mixture was heated slowly over a flame and shaken until a uniform solution was formed.

The liquid was allowed to cool to room temperature. The yield was quantitative and the product had a melting point of lower than room temperature. About 10 g of the synthesized deep eutectic solvent were put in a closed vial and kept in a dry space.

### 1-Mercapto-2-Propanol and Choline Chloride

Choline chloride and 1-mercapto-2-propanol were mixed in a 1:2 molar ratio. Choline chloride (0.1 mol) was employed as a dry powder and was added to 0.2 mol of 1-mercapto-2-propanol. The mixture was heated slowly over a flame and shaken until a uniform and clear solution was formed.

The liquid was allowed to cool to room temperature. The yield was quantitative and the product had a melting point of lower than room temperature. About 10 g of the synthesized deep eutectic solvent were put in a closed vial and kept in a dry space.

### Sodium 2-Mercaptoethansulfonate and Choline Chloride

Choline chloride and sodium 2-mercaptoethansulfonate were mixed in a 1:1 molar ratio. Choline chloride (0.1 mol) was employed as a dry powder and added to 0.1 mol of sodium 2-mercaptoethansulfonate. The mixture was heated slowly over a flame and shaken until a uniform solution had been formed.

The liquid was then centrifuged and the precipitate was discarded. About 2 g of the synthesized deep eutectic solvent were put in a closed vial and kept in a dry space.

### 4-Mercaptophenylacetic Acid and Choline Chloride

Choline chloride and 4-mercaptophenylacetic acid were mixed in a 1:2 molar ratio. Choline chloride (0.1 mol) was employed as a dry powder and was added to 0.2 mol of 4-mercaptophenylacetic acid. The mixture comprising was heated slowly over a flame and shaken. No eutecticum formed.

### 2-Methyl-2-Propanethiol and Choline Chloride

Choline chloride and 2-methyl-2-propanethiol were mixed in a 1:2 molar ratio. Choline chloride (0.1 mol) was employed as a dry powder and was added to 0.2 mol of 2-methyl-2-propanethiol. The mixture was heated slowly over a flame and shaken. No eutecticum formed.

### 2-,3-,10-Mercaptopinane and Choline Chloride

Choline chloride and 2-,3-,10-mercaptopinane were mixed in a 1:2 molar ratio. Choline chloride (0.1 mol) employed as a dry powder and was added to 0.2 mol of 2-,3-,10-mercaptopinane. The mixture as heated slowly over a flame and shaken. No eutecticum formed

### Example 4

Sortase Mediated Transamidation in Thiol-based Deep Eutectic Solvent

The molecules LCR640-ULPETGGRRC (LCR640 fluorophore conjugated to beta alanine (U); SEQ ID NO: 33) and GGG-biotin were dissolved in a thiol-based DES (choline chloride:dithiothreitol at molar ratio of 1:3) comprising 10% (v/v) water to a final concentration of 2 mM. In pure water LCR640-ULPETGGRRC solubilize to a concentration of less than 0.1 mM.

This solution was mixed with a solution comprising 1 mM Sa-SrtA (50 mM Tris-HCl pH 7.5, 150 mM NaCl, 10 mM CaCl) at a volume ratio of 19:1. The reaction mixture was incubated for 5 h at 37° C.

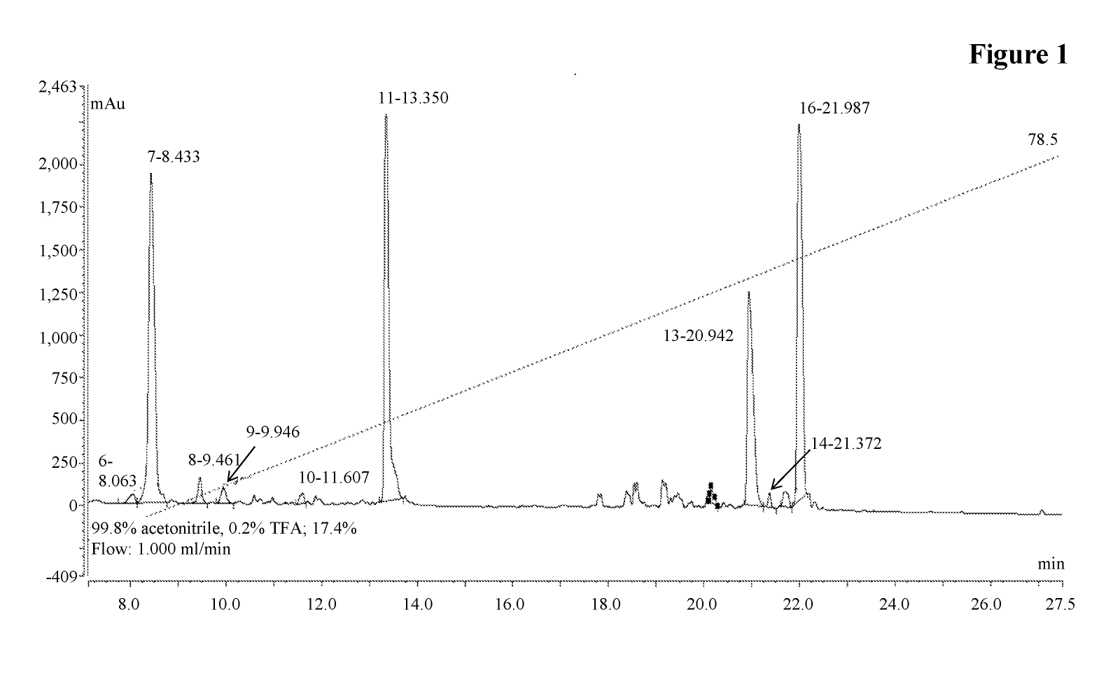

The chromatogram of the reaction products is shown in FIG. 1. The retention times of the compounds are showing in the following Table.

| retention time | |
|---|---|
| [min] | compound |
| 8.433 | GGG-biotin |
| 13.350 | Sa-SrtA |
| 20.942 | LCR640-LPETG |
| 21.987 | LCR640-LPETG-biotin |

The peak at 21.987 min. retention time corresponds to the reaction product.

1. A deep eutectic solvent consisting of (2-hydroxyethyl) trimethyl ammonium chloride and dithiothreitol in a molar ratio of from 1:2 to 1:3 and from more than 0% to 10% of co-solvent.

2. A deep eutectic solvent consisting of
a) i) (2-hydroxyethyl) trimethyl ammonium chloride, and
ii) 4-mercapto-1-butanol or 1-mercapto-2-propanol in a molar ratio of i) to ii) of about 1:2, and
b) of from more than 0% to about 10% co-solvent.

3. A deep eutectic solvent consisting of (2-hydroxyethyl) trimethyl ammonium chloride and sodium 2-mercaptoethansolfonate in a molar ratio of about 1:1 and of from more than 0% to about 10% co-solvent.

4. The deep eutectic solvent of claim 1, wherein the co-solvent is an aqueous co-solvent.

5. The deep eutectic solvent of claim 4, comprising from more than 0% up to about 5% (v/v) aqueous co-solvent.

6. A method for the enzymatic production of a polypeptide comprising the following step
incubating
i) a first polypeptide comprising the amino acid sequence LPXTG (SEQ ID NO: 01, wherein X can be any amino acid residue) or LPXTA (SEQ ID NO: 101, wherein X can be any amino acid residue),
ii) a second polypeptide comprising i) a glycinyl, an alaninyl, or a cysteinyl compound at its N-terminus, or ii) an oligoglycine, or oligoalanine, or a cysteine amino acid residue followed by one to three glycine or alanine amino acid residues at its N-terminus, or iii) a lysine amino acid residue within its 5 N-terminal amino acid residues, and
iii) a third polypeptide with sortase A activity,
in the deep eutectic solvent of claim 1;
wherein the polypeptide is produced.

7. The method of claim 6, wherein the second polypeptide has at its N-terminus the amino acid sequence GGG, AAA, CGG, CAA, KGG, or KAA (SEQ ID NO: 29, 162-166).

8. The method of claim 6, wherein the second polypeptide has at its N-terminus the amino acid sequence GGG or AAA.

9. The method of claim 6, wherein the first polypeptide comprises within the 25 C-terminal amino acid residues the amino acid sequence LPXTG (SEQ ID NO: 01, wherein X can be any amino acid residue) or LPXTA (SEQ ID NO: 101, wherein X can be any amino acid residue)

10. The method of claim 6, wherein the first polypeptide comprises at its C-terminus the amino acid sequence LPXTG (SEQ ID NO: 01, wherein X can be any amino acid residue) or LPXTA (SEQ ID NO: 101, wherein X can be any amino acid residue).

11. The method of claim 6, wherein the first polypeptide has at its C-terminus the amino acid sequence LPETG (SEQ ID NO: 04) or LPETA (SEQ ID NO: 108).

12. The method of claim 6, wherein the first polypeptide and the second polypeptide are independently of each other selected from an antibody variable domain, an antibody heavy chain Fab-fragment, an antibody Fc-region, a tag, and a peptide comprising the amino acid sequence LPXTG (SEQ ID NO: 01, wherein X can be any amino acid residue) or LPXTA (SEQ ID NO: 101, wherein X can be any amino acid residue), a linker and a non-sortase motif moiety.

13. The method of claim 6, wherein the third polypeptide comprises the amino acid sequence of SEQ ID NO: 05, SEQ ID NO: 06, SEQ ID NO: 27, SEQ ID NO: 156, SEQ ID NO: 159, SEQ ID No:160 or SEQ ID NO: 161.

14. The method of claim 6, wherein the third polypeptide has the amino acid sequence of SEQ ID NO: 05 or 06.


SEQUENCE LISTING (168 sequences)

SEQ ID NO: 1 - protein, 5 aa, Staphylococcus aureus
  MISC_FEATURE (3)..(3): X can be any amino acid residue
LPXTG

SEQ ID NO: 2 - protein, 4 aa, Artificial Sequence
  cystein sortase motif 1
CGGG

SEQ ID NO: 3 - protein, 4 aa, Artificial Sequence
  cystein sortase motif 2
CAAA

SEQ ID NO: 4 - protein, 5 aa, Artificial Sequence
  sortase motif
LPETG

SEQ ID NO: 5 - protein, 147 aa, Artificial Sequence
  Staphylococcus aureus Sortase A shortened version
QAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 6 - protein, 150 aa, Artificial Sequence
  Listeria monocytogenes Sortase A shortened version
ANYDKDAVVGSIAVPSVDVNLLVFKGTNTANLLAGATTMRSDQVMGKGNYPLAGHHMRDESMLFGPIMKVKKGDKIYLTDLENLYEYTVTETKTIDETEVSVIDDTKDARITLITCDKPTETTKRFVAVGELEKTEKLTKELENKYFPSK

SEQ ID NO: 7 - protein, 5 aa, Artificial Sequence
  Arg-tag
RRRRR

SEQ ID NO: 8 - protein, 6 aa, Artificial Sequence
  Arg-tag 2
RRRRRR

SEQ ID NO: 9 - protein, 6 aa, Artificial Sequence
  His-tag
HHHHHH

SEQ ID NO: 10 - protein, 19 aa, Artificial Sequence
  amino acid tag
KDHLIHNVHKEFHAHAHNK

SEQ ID NO: 11 - protein, 8 aa, Artificial Sequence
  amino acid tag
DYKDDDDK

SEQ ID NO: 12 - protein, 22 aa, Artificial Sequence
  amino acid tag
DYKDHDGDYKDHDIDYKDDDDK

SEQ ID NO: 13 - protein, 9 aa, Artificial Sequence
  amino acid tag#
AWRHPQFGG

SEQ ID NO: 14 - protein, 8 aa, Artificial Sequence
  amino acid tag
WSHPQFEK

SEQ ID NO: 15 - protein, 10 aa, Artificial Sequence
  amino acid tag
MDVEAWLGAR

SEQ ID NO: 16 - protein, 16 aa, Artificial Sequence
  amino acid tag
MDVEAWLGARVPLVET

SEQ ID NO: 17 - protein, 38 aa, Artificial Sequence
  amino acid tag
MDEKTTGWRGGHVVEGLAGELEQLRARLEHHPQGQREP

SEQ ID NO: 18 - protein, 10 aa, Artificial Sequence
  amino acid tag
EQKLISEEDL

SEQ ID NO: 19 - protein, 15 aa, Artificial Sequence
  amino acid tag
KETAAAKFERQHMDS

SEQ ID NO: 20 - protein, 26 aa, Artificial Sequence
  amino acid tag
KRRWKKNFIAVSAANRFKKISSSGAL

SEQ ID NO: 21 - protein, 47 aa, Artificial Sequence
  amino acid tag
PATTTGSSPGPTQSHYGQCGGIGYSGPTVCASGTTCQVLNPYYSQCL

SEQ ID NO: 22 - protein, 32 aa, Butyrivibrio fibrisolvens
MDWNANIAPGNSVEFGIQGAGSVGNVIDITVE

SEQ ID NO: 23 - protein, 51 aa, Artificial Sequence
  chitin-binding-domain
TNPGVSAWQVNTAYTAGQLVTYNGKTYKCLQPHTSLAGWEPSNVPALWQLQ

SEQ ID NO: 24 - protein, 209 aa, Chondrus crispus
MPEIKLTYFDMRGRAEASRLALVVGEIPFEDERVVFDHWKEAKPKTPYAALPMLTVDGMQVAQSDAILRYCGKLAGLYPSDPLEAAKVDEVGGVIDDVTHAMYRYRGDDKDKLREERDKFSKVDVPRYVGALEKRLEAFGDGPWAVGGNMTIADLHICHLVTNIRCGMLDFVDKDLLEGYVRIVKSYSAVMEHPKVTEWYEKKPVKMFS

SEQ ID NO: 25 - protein, 396 aa, Escherichia coli
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 26 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 27 - protein, 222 aa, Listeria monocytogenes
MLKKTIAAAALAAGLLLIFSPFIKNGIVKYMSGHETIEQYKASDIKKNNEKDATFDFESVQLPSMTSVIKGAANYDKDAVVGSIAVPSVDVNLLVFKGTNTANLLAGATTMRSDQVMGKGNYPLAGHHMRDESMLFGPIMKVKKGDKIYLTDLENLYEYTVTETKTIDETEVSVIDNTKDARITLITCDKPTETTKRFVAVGELEKTEKLTKELENKYFPSK

SEQ ID NO: 28 - protein, 2 aa, Artificial Sequence
  oligogylcine
GG

SEQ ID NO: 29 - protein, 3 aa, Artificial Sequence
  oligoglycine
GGG

SEQ ID NO: 30 - protein, 4 aa, Artificial Sequence
  oligoglycine
GGGG

SEQ ID NO: 31 - protein, 5 aa, Artificial Sequence
  oligoglycine
GGGGG

SEQ ID NO: 32 - protein, 12 aa, Artificial Sequence
  hexa-histidine tag with linker
MRGSHHHHHHGS

SEQ ID NO: 33 - protein, 11 aa, Artificial Sequence
  LCR640-ULPETGGGRRC
XLPETGGGRRC

SEQ ID NO: 34 - protein, 5 aa, Artificial Sequence
  sortase recognition motif
LPXTG

SEQ ID NO: 35 - protein, 4 aa, Artificial Sequence
  sortase recognition motif
LPXT

SEQ ID NO: 36 - protein, 5 aa, Artificial Sequence
  sortase recognition motif
LPTTG

SEQ ID NO: 37 - protein, 5 aa, Artificial Sequence
  sortase recognition motif
LPDTG

SEQ ID NO: 38 - protein, 5 aa, Artificial Sequence
  sortase recognition motif
LPKTG

SEQ ID NO: 39 - protein, 5 aa, Artificial Sequence
  sortase recognition motif
LPNTG

SEQ ID NO: 40 - protein, 5 aa, Artificial Sequence
  sortase recognition motif
PLAAG

SEQ ID NO: 41 - protein, 5 aa, Artificial Sequence
  sortase recognition motif
LPKAG

SEQ ID NO: 42 - protein, 5 aa, Artificial Sequence
  sortase recognition motif
LPQTG

SEQ ID NO: 43 - protein, 5 aa, Artificial Sequence
  sortase recognition motif
LPMTG

SEQ ID NO: 44 - protein, 5 aa, Artificial Sequence
  sortase recognition motif
LPITG

SEQ ID NO: 45 - protein, 5 aa, Artificial Sequence
  sortase recognition motif
LPDTA

SEQ ID NO: 46 - protein, 5 aa, Artificial Sequence
  sortase recognition motif
SPKTG

SEQ ID NO: 47 - protein, 5 aa, Artificial Sequence
  sortase recognition motif
LAETG

SEQ ID NO: 48 - protein, 5 aa, Artificial Sequence
  sortase recognition motif
LAATG

SEQ ID NO: 49 - protein, 5 aa, Artificial Sequence
  sortase recognition motif
LAHTG

SEQ ID NO: 50 - protein, 5 aa, Artificial Sequence
  sortase recognition motif
LASTG

SEQ ID NO: 51 - protein, 5 aa, Artificial Sequence
  sortase recognition motif
LPLTG

SEQ ID NO: 52 - protein, 5 aa, Artificial Sequence
  sortase recognition motif
LSRTG

SEQ ID NO: 53 - protein, 5 aa, Artificial Sequence
  sortase recognition motif
VPDTG

SEQ ID NO: 54 - protein, 5 aa, Artificial Sequence
  sortase recognition motif
IPQTG

SEQ ID NO: 55 - protein, 5 aa, Artificial Sequence
  sortase recognition motif
YPRTG

SEQ ID NO: 56 - protein, 5 aa, Artificial Sequence
  sortase recognition motif
LALTG

SEQ ID NO: 57 - protein, 5 aa, Artificial Sequence
  sortase recognition motif
LGNTG

SEQ ID NO: 58 - protein, 5 aa, Artificial Sequence
  sortase recognition motif
LPQTS

SEQ ID NO: 59 - protein, 5 aa, Artificial Sequence
  sortase recognition motif
TLXTC

SEQ ID NO: 60 - protein, 5 aa, Artificial Sequence
  sortase recognition motif
NPQTN

SEQ ID NO: 61 - protein, 5 aa, Artificial Sequence
  sortase recognition motif
XPXXG

SEQ ID NO: 62 - protein, 5 aa, Artificial Sequence
  sortase recognition motif
IPKTG

SEQ ID NO: 63 - protein, 5 aa, Artificial Sequence
  sortase recognition motif
IPALG

SEQ ID NO: 64 - protein, 5 aa, Artificial Sequence
  sortase recognition motif
LAASS

SEQ ID NO: 65 - protein, 5 aa, Artificial Sequence
  sortase recognition motif
LAPTG

SEQ ID NO: 66 - protein, 5 aa, Artificial Sequence
  sortase recognition motif
LPISS

SEQ ID NO: 67 - protein, 5 aa, Artificial Sequence
  sortase recognition motif
LPKTS

SEQ ID NO: 68 - protein, 6 aa, Artificial Sequence
  sortase recognition motif
YALPET

SEQ ID NO: 69 - protein, 8 aa, Artificial Sequence
  sortase recognition motif
YALPMTGK

SEQ ID NO: 70 - protein, 4 aa, Artificial Sequence
  sortase recognition motif
LPXA

SEQ ID NO: 71 - protein, 4 aa, Artificial Sequence
  sortase recognition motif
SPXT

SEQ ID NO: 72 - protein, 4 aa, Artificial Sequence
  sortase recognition motif
LAXT

SEQ ID NO: 73 - protein, 4 aa, Artificial Sequence
  sortase recognition motif
LSXT

SEQ ID NO: 74 - protein, 4 aa, Artificial Sequence
  sortase recognition motif
NPXT

SEQ ID NO: 75 - protein, 4 aa, Artificial Sequence
  sortase recognition motif
VPXT

SEQ ID NO: 76 - protein, 4 aa, Artificial Sequence
  sortase recognition motif
IPXT

SEQ ID NO: 77 - protein, 4 aa, Artificial Sequence
  sortase recognition motif
LGXT

SEQ ID NO: 78 - protein, 4 aa, Artificial Sequence
  sortase recognition motif
YPXR

SEQ ID NO: 79 - protein, 4 aa, Artificial Sequence
  sortase recognition motif
LPST

SEQ ID NO: 80 - protein, 4 aa, Artificial Sequence
  sortase recognition motif
LPKT

SEQ ID NO: 81 - protein, 4 aa, Artificial Sequence
  sortase recognition motif
LPIT

SEQ ID NO: 82 - protein, 4 aa, Artificial Sequence
  sortase recognition motif
LPDT

SEQ ID NO: 83 - protein, 4 aa, Artificial Sequence
  sortase recognition motif
SPKT

SEQ ID NO: 84 - protein, 4 aa, Artificial Sequence
  sortase recognition motif
LAET

SEQ ID NO: 85 - protein, 4 aa, Artificial Sequence
  sortase recognition motif
LAAT

SEQ ID NO: 86 - protein, 4 aa, Artificial Sequence
  sortase recognition motif
LAST

SEQ ID NO: 87 - protein, 4 aa, Artificial Sequence
  sortase recognition motif
LPLT

SEQ ID NO: 88 - protein, 4 aa, Artificial Sequence
  sortase recognition motif
LSRT

SEQ ID NO: 89 - protein, 4 aa, Artificial Sequence
  sortase recognition motif
LPET

SEQ ID NO: 90 - protein, 4 aa, Artificial Sequence
  sortase recognition motif
VPDT

SEQ ID NO: 91 - protein, 4 aa, Artificial Sequence
  sortase recognition motif
IPQT

SEQ ID NO: 92 - protein, 4 aa, Artificial Sequence
  sortase recognition motif
YPRR

SEQ ID NO: 93 - protein, 4 aa, Artificial Sequence
  sortase recognition motif
LPMT

SEQ ID NO: 94 - protein, 4 aa, Artificial Sequence
  sortase recognition motif
LAFT

SEQ ID NO: 95 - protein, 4 aa, Artificial Sequence
  sortase recognition motif
LPQT

SEQ ID NO: 96 - protein, 4 aa, Artificial Sequence
  sortase recognition motif
XPXX

SEQ ID NO: 97 - protein, 5 aa, Artificial Sequence
  sortase recognition motif
YPPRG

SEQ ID NO: 98 - protein, 5 aa, Artificial Sequence
  sortase recognition motif
LAFTG

SEQ ID NO: 99 - protein, 5 aa, Artificial Sequence
  sortase recognition motif
LPXAG

SEQ ID NO: 100 - protein, 5 aa, Artificial Sequence
  sortase recognition motif
LPNAG

SEQ ID NO: 101 - protein, 5 aa, Artificial Sequence
  sortase recognition motif
LPXTA

SEQ ID NO: 102 - protein, 5 aa, Artificial Sequence
  sortase recognition motif
LPNTA

SEQ ID NO: 103 - protein, 5 aa, Artificial Sequence
  sortase recognition motif
LGXTG

SEQ ID NO: 104 - protein, 5 aa, Artificial Sequence
  sortase recognition motif
LGATG

SEQ ID NO: 105 - protein, 5 aa, Artificial Sequence
  sortase recognition motif
IPXTG

SEQ ID NO: 106 - protein, 5 aa, Artificial Sequence
  sortase recognition motif
IPNTG

SEQ ID NO: 107 - protein, 5 aa, Artificial Sequence
  sortase recognition motif
IPETG

SEQ ID NO: 108 - protein, 5 aa, Artificial Sequence
  sortase recognition motif
LPETA

SEQ ID NO: 109 - protein, 5 aa, Artificial Sequence
  sortase recognition motif
LPXTX

SEQ ID NO: 110 - protein, 5 aa, Artificial Sequence
  sortase recognition motif
LPXTA

SEQ ID NO: 111 - protein, 5 aa, Artificial Sequence
  sortase recognition motif
LPXTG

SEQ ID NO: 112 - protein, 85 aa, Streptomyces coelicolor
VAGHVDNAEGPAVFYRLGALEKGSAIEIDRRDGGVAVFTVDAVEVYAADAFPDEKVYGAADRPELRVITCGGPYSRSTGYQGNVV

SEQ ID NO: 113 - protein, 85 aa, Streptomyces coelicolor
VVGHVDNQQGPAVFYGLGALKKGNKVEVHRQDGKTAVFEIYGIEVFEKNNFPGDRVYGSKGSPELRVITCGGGFTKQNGYDGNVV

SEQ ID NO: 114 - protein, 86 aa, Streptomyces coelicolor
IAGHVDTKTSAAVFARLDQLDKGDKFQVRRADGRSATFVVDGLETFAKDEFPSDRVYGDADRPEVRLITCAGDYDHKVKDYTDNLV

SEQ ID NO: 115 - protein, 88 aa, Streptomyces coelicolor
MVGHVDTETRPAVFYQLSTLEPGQTIRVARDDDEVAEFTVDDVQVLTRDGFDAQQAYGPRDTGRSELRLITCGGTFDQTTDSYTANVV

SEQ ID NO: 116 - protein, 78 aa, Bacillus halodurans
LSGHRDTVFRDMGKLEIGDDLTVHMPYGSYTYRIVDTEIVDANDTSVIRSTAPDEVLTLSTCYPFNFIGSAPERYIIY

SEQ ID NO: 117 - protein, 78 aa, Bacillus subtilis
LSGHRDTVFRRTGELEKGDQLRLLLSYGEFTYEIVKTKIVDKDDTSIITLQHEKEELILTTCYPFSYVGNAPKRYIIY

SEQ ID NO: 118 - protein, 77 aa, Bacillus halodurans
LSGHRDTVFRELGEVGVGDLLIVETATGTHTYRVRKVRIVDEDDRTVIVPKPRATLTVSTCYPFDFIGSAPERYILE

SEQ ID NO: 119 - protein, 77 aa, Bacillus anthracis
LSGHRDTVFTDLGQLKEKDTLVLEYDNKTYTYEIQKIWITHADDRTVIIKKEEPILTLTTCYPFDYLGDAPDRYLIB

SEQ ID NO: 120 - protein, 81 aa, Bacillus halodurans
IAAHRSRTYGRQPNRLDEVEGDVITVTTNNHMYRYTVYSITVVEPTNIDILQHDGTAPVLTLLTCDPVKDPTHRLIVQAEM

SEQ ID NO: 121 - protein, 81 aa, Clostridium acetobutylicum
LAGHRSYTFGEYFNRLGEIGSGDEIDVETVNGTFKYKVYSTKVVLPSEVHVLDQTKDPTMTLVTCTPIRIATHRLIIKAKR

SEQ ID NO: 122 - protein, 89 aa, Streptomyces coelicolor
LAGHRNTHGEPFRYINKLEPGDPIVVETQDKYFVYKMASILPVTSPSNVSVLDPVPKQSGFKGPGRYITLTTCTPEFTSKYRMIVNGKM

SEQ ID NO: 123 - protein, 89 aa, Streptomyces coelicolor
LAAHRDGHGARFHNIDKIEKGDPIVFETKDTWYVYKTYAVLPETSKYNVEVLGGIPKESGKKKAGHYITLTTCTPVYTSRYRYVVWGEL

SEQ ID NO: 124 - protein, 81 aa, Actinomyces naeslundii
ITGHRGLAEATMFTNLDKVTGDSLIVEVFGEVLTYRVTSTKVVEPEETEALRVEEGKDLLTLVTCIPLGINTHRILLTGER

SEQ ID NO: 125 - protein, 81 aa, Corynebacterium diphtheriae
ITAHRGLAEATMFTNLNKVGVGDRFTIEWGEVLTIEVRETRVVSPEDTRFLQTQDDRDLVILVTCLPLGINTHRILVTAER

SEQ ID NO: 126 - protein, 82 aa, Streptococcus pneumoniae
ITAPTGLPTAKMFTDLTKLKVGDKFYVHNIKEVMAYQVDQVKVIEPTNFDDLLIVPGHDYVTLLTCTPYMINTHRLLVRGHR

SEQ ID NO: 127 - protein, 82 aa, Streptococcus pneumoniae
ITHARGLPTAELFSQLDKMKKGDIFYLHVLDQVLAYQVDQIVTVEPNDFEVLIQHGEDAYATLLTCTPYMINSHRLLVRGKR

SEQ ID NO: 128 - protein, 83 aa, Enterococcus faecalis
ISSGHRGLPQAKLFTDLPELKKGDEFYIEVNGKTLAYQVDQIKTVEPTDTKDLHIESGQDLVTLLECTPYMINSHRLLVRGHR

SEQ ID NO: 129 - protein, 80 aa, Streptococcus equinus
ISGHRGLPSAKLFTNIDKLRINDTFTTSLNRTMTYQIDKATVLPDDVSLLRIEEGKDLVTLVTCTPYGVNTHRLLVRGHR

SEQ ID NO: 130 - protein, 80 aa, Streptococcus pyogenes
ISANRGLPAEMFTNLNLVKKGDTFYFRVLNKVLAYKVDQILTTVEPDQNSLSQVMGKDATLVTCTPYGVNTKRLLVRGHR

SEQ ID NO: 131 - protein, 81 aa, Corynebacterium diphtheriae
ITGHSGLANATLFDLEDVKSHDPIYITVQGETLKYEVDAINVVLPEDTKLLAPDPNKDQITLITCTFYAVNSHRLLVRAHR

SEQ ID NO: 132 - protein, 82 aa, Corynebacterium diphtheriae
ITGHTGLANSIMFDHLNYAEKGDIFYVQVSGEKLKYVVDQIKVVLPTETEDLRPEQGKDYILLITCTPYGINTHRLMVRGHQ

SEQ ID NO: 133 - protein, 80 aa, Corynebacterium diphtheriae
LSAHTGLQNATLWDNLIQIKKGDPVYVAAGEKLKYEVRNIEVVTPDKTSLLRRTSNKDQVTLITCTPYGINTHRLIIAER

SEQ ID NO: 134 - protein, 81 aa, Corynebacterium diphtheriae
LTAHSGEQKSTFFDNLEKVKKGDAIYVRNIGETLKYQVRDIEIIRPAEEIDRIQFPDRDLITLVECPYGINTHRLLVTAER

SEQ ID NO: 135 - protein, 74 aa, Bacillus halodurans
IAGHRGYRGNRHFSRLPDVTIGDEVFLHTKETFDISIIEPTDVDVLDDRDGKHEITMITCTRSGKQRVAVRGEL

SEQ ID NO: 136 - protein, 80 aa, Bacillus halodurans
IAGHRGYRGNRHFSRLPDVTIGDEVFLHTKEETFVYKVTDISIIEPTDVDILDDRDGKHEITMITCTRSGKQRVAVRGVL

SEQ ID NO: 137 - protein, 85 aa, Streptococcus mutans
LASHHVPGMTGSSQMLFSPLERAKEGMEIYLTDKNKVYTYVISEKTVTPEHVEVIDNRPGQNEVTLVTCTDAGATARTIVHGTYK

SEQ ID NO: 138 - protein, 88 aa, Streptococcus pyogenes
LASHHIFPGITGSSQTFLFSPLERAQNGMSIYLTDKBKIYEYIIKDVPIVAPERVDVIDDTAGLKEVLLVRCTDIEATERIIVKGELK

SEQ ID NO: 139 - protein, 85 aa, Streptococcus pneumoniae
LASHHIFGVDNANKMLFSPLDNAKNGMKIYLTDKNKVYTYEIREVKRVTPDRVDEVDDRDGVNEITLVTCELAATERIIVKGDLK

SEQ ID NO: 140 - protein, 82 aa, Enterococcus faecalis
LASHRTEDGVSLFSPLERTKKDELIYITDLSTVYTYKITSVEKIEPTRVELIDDVPGQNMITLITCGDLQATTRIAVQGTLA

SEQ ID NO: 141 - protein, 90 aa, Bacillus halodurans
DHHEGFYYDTLYNRYDVEVFSAYVTTTDFYYIETEFPSKDDYKAFLNELKKRSVVQTNVEVGEEDQIITLSTCDYRLDRDRGRLVVHGKL

SEQ ID NO: 142 - protein, 91 aa, Bacillus anthracis
FMSHRKLYYDTLFEGYDLEVFSVYTTTTDFYYIETDFSSDTEYTSFLEKIQEKSLYKTDTTVTAGDQIVTLSTCDYALDPEAGRLVVHAKL

SEQ ID NO: 143 - protein, 91 aa, Staphylococcus aureus
YEKHKIIEPDNKYGKYQLQVYFSAYKTTTKDNYIRTDFENDQDYQQFLDETKRKSVINSDVNVTVKDKIMTLSTCEDAYSTTKRIVVVAKI

SEQ ID NO: 144 - protein, 92 aa, Streptococcus pyogenes
FNKHKEPSIETKTKQKLKINIFACIQTDAFDSLLFNPIDVDISSKNEFLTNHIKQKSVQYREILTTNESRFVALSTCEDMTTDGRIIVIGQI

SEQ ID NO: 145 - protein, 91 aa, Streptococcus pyogenes
FNKHNKAIIETKERKKLTVTIFACLKTDAFDQLVFNPNAITNQDQQKQLVDYISKRSKQFKPVKLKHHTKFVAFSTCENSTDNRVIVVGTI

SEQ ID NO: 146 - protein, 81 aa, Streptococcus pneumoniae
IAGHRAEPSHVFFRHLDQLKVGDALYYDNGQEIVEYQMMDTEIILPSEWEKLESVSSKNIMTLTCDPIPTFNKRLLVNFER

SEQ ID NO: 147 - protein, 77 aa, Bacillus anthracis
LAGHNMSKKGVLFSDIASLKKGDKIYDNENEYEYAVTGVSEVTFDKWEVVEDHGKDELITCVSVKDNSKRYVVAGDL

SEQ ID NO: 148 - protein, 81 aa, Bacillus subtilis
LAGHHLKQKNLLFGPLENIKTGAQIVITDFKKDYIYSVTSKDIISEMDADVVEETNKKEITLITCDKAVKTEGRLVVKGEL

SEQ ID NO: 149 - protein, 81 aa, Enterococcus faecalis
LASHNAGYEGLLFTSLNKVSVGDLVKLNDRGHSFIYKVKEQKHVDMTDTTMLNLTRKPTLTLITCDQATKTTGRIIVIAEL

SEQ ID NO: 150 - protein, 84 aa, Staphylococcus aureus
IAGHTFIDRPNYQFTNLKAAKKGSMVYFKVGNETRKYKMTSIRDVKPTDVGVLDEQKGKDKQLTLITCDDYNEKTGVWEKRKIF

SEQ ID NO: 151 - protein, 79 aa, Shewanella putrefaciens
IAGHRDTHFAILKGMTVGRRLALQTAAGKEIVYQVVATKESQTELMAPSDDNRLTLITCYPPDALQGVAELRFVVQAVP

SEQ ID NO: 152 - protein, 91 aa, Streptomyces coelicolor
VLLGHVTVGRYGDGVFRHLAGLRRGERIEARLENGTTAEFTVTAVRTVAKADFPTDDVYGDVAGPELRLITCGGPRDGQEYRDNVIVFAEL

SEQ ID NO: 153 - protein, 90 aa, Clostridium difficile
IYGHNMKNKTMFNNLNKFKDADFFKKNNKIKITLNGREFLYDVFSAYIVESDYDYLKTNFNNESDYQNYNDITSKSLYKSPIKVNSNDKI

SEQ ID NO: 154 - protein, 86 aa, Methanothermobacter thermautotrophicus
ILGHRTTYSGPFRKIGALRKGDRVIIEDASSSIRYIYTVTSNGDDIRWDYRTNPVRFSQSGDARLMLITCYPPGQKKAAWITHCKL

SEQ ID NO: 155 - protein, 147 aa, Artificial Sequence; identical to SEQ ID NO: 5
  Staphylococcus aureus Sortase A shortened version N(1-59)

SEQ ID NO: 156 - protein, 168 aa, Artificial Sequence
  Streptococcus pyogenes Sortase A shortened version
VLQAQMAAQQLPVIGGIAIPELGINLPIFKGLGNTELIYGAGTMKEEQVMGGENNYSLASHHIFGITGSSQMLFSPLERAQNGMSIYLTDKEKIYEYIIKDVFTVAPERVDVIDDTAGLKEVTLVTCTDIEATERIIVKGELKTEYDFDKAPADVLKAFNHSYNQVST

SEQ ID NO: 157 - protein, 206 aa, Staphylococcus aureus; identical to SEQ ID NO: 26

SEQ ID NO: 158 - protein, 222 aa, Listeria monocytogenes; identical to SEQ ID NO: 27

SEQ ID NO: 159 - protein, 249 aa, Streptococcus pyogenes
MVKKQKRRKIKSMSWARKLLIAVLLILGLALLFNKPIRNTLIARNSNKYQVTKVSKKQIKKNKEAKSTFDFQAVEPVSTESVLQAQMAAQQLPVIGGIAIPELGINLPIFKGLGNTELIYGAGTMKEEQVMGGENNYSLASHHIFGITGSSQMLFSPLERAQNGMSIYLTDKEKIYEYIIKDVFTVAPERVDVIDDTAGLKEVTLVTCTDIEATERIIVKGELKTEYDFDKAPADVLKAFNHSYNQVST

SEQ ID NO: 160 - protein, 178 aa, Artificial Sequence
  Staphylococcus aureus Sortase A shortened version N(2-29)
MDNYLHDKDKDEKIEQYDKNVKEQASKDKKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 161 - protein, 222 aa, Listeria monocytogenes
MLKKTIAAAALAAGLLLIFSPFIKNGIVKYMSGHETIEQYKASDIKKNNEKDATFDFESVQLPSMTSVIKGAANYDKDAVVGSIAVPSVDVNLLVFKGTNTANLLAGATTMRSDQVMGKGNYPLAGHHMRDESMLFGPIMKVKKGDKIYLTDLENLYEYTVTETKTIDETEVSVIDDTKDARITLITCDKPTETTKRFVAVGELEKTEKLTKELENKYFPSK

SEQ ID NO: 162 - protein, 3 aa, Artificial Sequence
  sortase tag
AAA

SEQ ID NO: 163 - protein, 3 aa, Artificial Sequence
  sortase tag
CGG

SEQ ID NO: 164 - protein, 3 aa, Artificial Sequence
  sortase tag
CAA

SEQ ID NO: 165 - protein, 3 aa, Artificial Sequence
  sortase tag
KGG

SEQ ID NO: 166 - protein, 3 aa, Artificial Sequence
  sortase tag
KAA

SEQ ID NO: 167 - protein, 206 aa, Staphylococcus aureus; identical to SEQ ID NO: 26

SEQ ID NO: 168 - protein, 222 aa, Listeria monocytogenes; identical to SEQ ID NO: 27